SEQUENCE LISTING (1 sequence)

SEQ ID NO: 1 - protein, 7 aa, Artificial Sequence
  Synthetic
GGGVXED

(19) United States
(12) Patent Application Publication
(10) Pub. No.: US 2016/0279108 A1
(43) Pub. Date: Sep. 29, 2016

(54) TARGETED MTOR INHIBITORS

(71) Applicants: MARCUS LAIRD FORREST, LAWRENCE, KS (US); TI ZHANG, LAWRENCE, KS (US); S P SANJEEWA N. SENADHEERA, LAWRENCE, KS (US); DANIEL AIRES, MISSION HILLS, KS (US); YUNQI ZHAO, CHENGGONG (CN); SHAOFENG DUAN, LAWRENCE, KS (US); CHAD GROER, LAWRENCE, KS (US)

(72) Inventors: MARCUS LAIRD FORREST, LAWRENCE, KS (US); TI ZHANG, LAWRENCE, KS (US); S P SANJEEWA N. SENADHEERA, LAWRENCE, KS (US); DANIEL AIRES, MISSION HILLS, KS (US); YUNQI ZHAO, CHENGGONG (CN); SHAOFENG DUAN, LAWRENCE, KS (US); CHAD GROER, LAWRENCE, KS (US)

(73) Assignees: UNIVERSITY OF KANSAS, LAWRENCE, KS (US); NANOPHARM LLC (d/b/a HYLAPHARM), LAWRENCE, KS (US)

(21) Appl. No.: 15/052,302

(22) Filed: Feb. 24, 2016

Related U.S. Application Data
(60) Provisional application No. 62/120,215, filed on Feb. 24, 2015.

(51) Int. Cl. A61K 31/436; A61K 47/48

(52) U.S. Cl. CPC A61K 31/436; A61K 47/48338; A61K 47/48923; A61K 47/4823; A61K 47/48023

(57) ABSTRACT

The present invention is directed to drug conjugates of mTOR inhibitors comprising an mTOR inhibitor, such as rapamycin, conjugated to hyaluronic acid by a linker comprising an ester, carbonate, or carbamate. The present invention is also directed to pharmaceutical compositions comprising the drug conjugates, and methods of making and using the drug conjugates and the pharmaceutical compositions.

### CROSS-REFERENCE TO RELATED APPLICATIONS

This application is based on and claims priority to U.S. Provisional Application Ser. No. 62/120,215, filed on Feb. 24, 2015, which is incorporated herein by reference in its entirety.

### STATEMENT REGARDING FEDERALLY SPONSORED RESEARCH OR DEVELOPMENT

This invention was made with government support under 1R01CA173292-01 awarded by the National Institutes of Health. The government has certain rights in the invention.

### INCORPORATION OF SEQUENCE LISTING

A paper copy of the Sequence Listing and a computer readable form of the Sequence Listing containing the file named “14KU058L_ST25.txt”, which is 654 bytes in size (as measured in MICROSOFT WINDOWS® EXPLORER), are provided herein and are herein incorporated by reference. This Sequence Listing consists of SEQ ID NO:1.

### BACKGROUND OF THE INVENTION

1. Field of the Invention

The present invention is directed to targeted drug conjugates, specifically targeted conjugates of mTOR inhibitors.

2. Background of the Invention

Rapamycin is a selective inhibitor of mammalian target of rapamycin (mTOR) and blocks the subsequent activation of p70 S6 kinase. The mTOR pathway regulates a variety of cellular signals and development processes, including mitogenic growth factors, hormones, nutrients, cellular energy levels and stress conditions. It is frequently activated in certain human cancers, such as breast cancer. The mTOR pathway is also widely involved in both apoptotic and autophagic pathways during oxidative stress. Therefore, inhibiting the mTOR pathway is extensively considered as an effective approach for targeted cancer therapy.

Rapamycin belongs to a class of immunomodulatory agents that are relatively specific and noncytotoxic on the immune system compared to other immunomodulators, such as tacrolimus and cyclosporine A. Being a macrocyclic immunosuppressive agent, rapamycin becomes active only when bound to mTOR.

Rapamycin blocks the cytokine-mediated signal transduction pathways during T-cell cycle progression, which results in the modulation of activity of a target protein by the rapamycin: FKBP complex sirolimus effector protein. Rapamycin has demonstrated inhibitory effects on activation of p70S6 kinase, activation of cdk2/cyclin E complex, phosphorylation of retinoblastoma protein, and suppression of cdc2 and cyclin A transcription.

In addition, a substantial and growing body of evidence suggests that within heterogeneous tumor populations a definable sub-population of cells drive tumor initiation, growth, dissemination, and recurrence. This “stem cell model” suggests that cancers originate and propagate from cancer stem cells (CSCs). CSCs occur in different proportions depending on the specific tumor type, but bear the capacity to self-renew and generate non-CSC, differentiated progeny. Operationally, CSCs are defined as cells with the ability to propagate new tumors. They may initiate tumor growth at the same site after surgical resection of a primary tumor (relapse) or at other locations (metastasis); only a very small number of CSCs are required to initiate a viable tumor in vivo. CSCs have been described for many types of cancers, including breast, leukemia, brain, colon, lung, and prostate.

CSCs relative quiescence and resistance to chemotherapy and radiation can render them resistant to agents that effectively kill the bulk of a tumor mass. Such resistance necessitates careful selection of drug targets. The PI3K/PTEN/Akt/mTOR pathway is involved in cell survival and inhibition of apoptosis, and activation of this pathway has been implicated in the pathogenesis of malignancies, including metastasis and resistance to cancer therapies. It has been observed that PI3K/PTEN/Akt/mTOR pathway was specifically activated in a sub-population of MCF7 cells that displayed enhanced colony formation in vitro and tumorigenicity in vivo. Further, inhibition of mTOR with rapamycin reduced stem cell proliferation in leukemia models, while sparing hematopoietic stem cells, suggesting that it may be possible to selectively target cancer stem cells without unduly harming healthy stem cells. Further, aberrant up-regulation of this pathway is estimated to occur in greater than 70% of breast cancers, and its inhibition has been effective in breast cancers that are resistant to hormonal therapies.

These data suggest that inhibition of the PI3K/PTEN/Akt/mTOR pathway may be an effective way of killing CSCs. Although many compounds inhibit various steps in this pathway, the most extensively studied are the mTOR inhibitors, such as rapamycin and its analogs (aka rapalogs). Importantly, rapalogs have demonstrated reasonable efficacy and safety profiles in clinical trials as both single agents and in combination therapies.

Though effective in human transplantation, systemic administration of rapamycin has considerable side effects. Common side effects include development of interstitial pneumonitis, increased serum lipids, decreased hemoglobin, arthralgia, peripheral edema, skin disorders, stomatitis, electrolyte disturbances (e.g. hypokalemia and hypophosphatemia), dyspnea, cough, infectious diseases and a higher incidence of lymphoceles. In addition, immunosuppressants have been indicated to increase the risk of cancer after use.

Because of rapamycin's extremely poor solubility and poor chemical stability, it is difficult to formulate for parenteral administration. Carrier molecules can be used to improve the solubility of poorly soluble cytotoxic agents. Abraxis Bioscience developed a serum albumin adsorbed formulation of the poorly soluble potent cytotoxic paclitaxel, which was successful in the clinic at reducing toxicity and improving efficacy. The same technology was applied to rapamycin to produce nab-rapamycin, but this has failed to be adopted clinically due to limited efficacy. Despite the advantage that would be expected by developing a safe soluble formulation of this poorly soluble cytotoxic, the failure of nab-rapamycin in the clinic shows that simply solubilizing poorly soluble cytotoxics is often insufficient to treat disease.

Several rapalogs with improve solubility or dispersibility have also been reported, e.g. temsirolimus, everolimus and deforolimus. With the exception of temsirolimus and everolimus, these have not been successful in treatment of cancers, and temsirolimus and everolimus have been limited in efficacy to renal cancers. This again shows that analogues that have improved solubility or chemical modification may not solve clinical problems with their use and be effective in cancer. In addition, the more soluble temsirolimus and everolimus were associated with high risks of fatal adverse events, showing that improving the solubility and formulation ability of rapalogs can lead to unexpected toxicity.

The failure of soluble formulations of cytotoxics is often due to wide distribution of these lipophilic compounds in the body, wherein they distribute throughout tissues without concentrating sufficiently in the diseased tissues, leading to non-specific toxicities and little efficacy. For a few cancers, targeting agents have been identified, such as trastuzumab for Her2 positive breast cancers. However, clinical failures of other antibodies demonstrates that targeting is not an obvious solution for many cancers. Gemtuzumab ozogamicin (Mylotarg) was designed to target leukemia with high affinity and to deliver a high potency cytotoxic drug. It was initially approved by the FDA under an accelerated process, but it was then withdrawn in 2010 after it failed to meet efficacy goals in larger post-approval trials.

Despite the success of targeted drug carriers such as Gemtuzumab ozogamicin and brentuximab vedotin to treat disease with fewer side effects, a reduction in toxicity is not obvious with targeting. 99mTc-fanolesomab (NeutroSpec) was initially approved by the FDA, but then withdrawn in 2005 after serious side effects including several patient deaths. Targeted therapies even without attached drugs can have unanticipated side effects. TeGenero's anti-CD28 therapy cause a patient to go into a coma for 3 weeks with heart, liver and renal failure after a single dose in a phase I trial. Although targeted carriers are known, all parts of a targeted carrier, including the targeting moiety, any carrier and/or linker and the drug can lead to failure of a targeted carrier due to lack of efficacy and serious side effects.

### BRIEF SUMMARY OF THE INVENTION

One aspect of the present invention is directed to a pharmaceutical composition comprising a drug conjugate, wherein the drug conjugate comprises an mTOR inhibitor selected from the group consisting of rapamycin, everolimus, temsirolimus, and deforolimus, a hyaluronic acid, and a linker coupling the hyaluronic acid to the mTOR inhibitor, wherein the linker comprises an ester, a carbonate, or a carbamate coupled to the mTOR inhibitor. In certain embodiments, the linker further comprises an amide, hydrazide, ester, carbonate, ether, carbamate, carbonyl, urea, alkyl or amine coupled to the hyaluronic acid. In certain aspects, the linker comprises amino acid that provides an amino group for an amide linkage with hyaluronic acid.

In certain embodiments wherein the linker comprises the ester, the carbonate or the carbamate the linker also comprises a hindered or electron rich labile bond. The linker may comprise an aromatic group, such as benzene. In one aspect of the invention the linker comprises an ester comprises 3-amino-4-methoxy-benzoate, and wherein a 3-amino group forms the amide.

In certain embodiments of the invention, the linker comprises a carbamate, which may be a comprised of a diamine. In one aspect of the invention, the linker comprises 1, 4-butanediamine.

In certain embodiments, the linker comprises a biologically labile linkage that is preferentially cleaved inside cells, wherein said cleavage results in spontaneous labiality of the ester, carbamate, or carbonate. In certain aspects of the invention, the biologically labile linkage is a biologically labile peptide sequence or a biologically labile disulfide linkage.

In embodiments where the linker comprises a biologically labile peptide sequence includes at least one sequence selected from the group consisting of Phe-Lys, Val-Lys, Ala-Lys, Phe-Phe-Lys, Ala-Phe-Lys, Gly-Phe-Lys, Ac-Phe-Lys, HCO-Phe-Lys, Val-Cit, Phe-Cit, Leu-Cit, Ile-Cit, Trp-Cit, Phe-Arg(NO₂)₂, and Phe-Arg(Ts). In certain aspects, the peptide sequence comprises Val-Cit, and can comprise Gly-Gly-Gly-Val-Cit-Glu-Asp.

In embodiments wherein the biologically labile sequence is a biologically labile disulfide linkage, the disulfide linkage may comprise an ethyl or propyl thiol group, such as an ethyldisulfide. In certain embodiments wherein the linker comprises an ester, carbonate or carbamate, the cleavage inside the cell can results in formation of a 5 or 6 member ring able to induce lability in the ester, carbonate or carbamate. In certain aspects, linker comprises an amino acid having an amino group, and the amino group provides an amide linkage with hyaluronic acid.

In certain aspects of the invention, the conjugate is a nanoparticle configured for preferential uptake into a tumor or lymph node. The nanoparticle may have a size between 9 and 100 nm.

The present invention is further directed to a therapeutic method comprising administering a therapeutically effective amount of any of the compositions the present invention to a subject in need thereof.

In certain embodiments, the present invention is directed to a pharmaceutical composition comprising a drug conjugate where the drug conjugate comprises an mTOR inhibitor selected from the group consisting of BGT226, SF1126, BEZ235, Gedatolisib and SF1101, hyaluronic acid, and a linker coupling the hyaluronic acid to the mTOR inhibitor, wherein the linker comprises an ester, a carbonate, or a carbamate coupled to the mTOR inhibitor.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 depicts a synthesis scheme of (A) HA-Temsirolimus and (B) 42-(3′-amino-4′methoxy)benzoate (HA-L-Rapa).

FIG. 2 (A) depicts DSC profiles of rapamycin, HA₃₅ₖ and different loading degree HA-L-Rapa and FIG. 2 (B) TGA analysis of HA_{35K} and different loading degree HA-L-Rapa.

FIG. 3 depicts the results of flow cytometry analysis of available CD44 receptor binding sites on the cell surface in which (A) depicts MDA-MB-468 and (B) depicts MDA-MB-468 treated with H-CAM, in each case stained with PE anti-CD44 antibody (dark grey) and PE IgGI isotope (light grey).

FIG. 4 depicts the results of a cell viability assay of rapamycin and HA-L-Rapa in MDA-MB-468 cells (A) CD44 positive cells and (B) with H-CAM blocking of CD44 (Mean±SD) (*, p<0.05).

FIG. 5 depicts plasma rapamycin concentration versus time disposition (Mean±SD, n=3).

FIG. 6 depicts HA-L-Rapa (A) animal survival and (B) suppressed tumor progression in BALB/c mice with 4T1.2neu breast cancer (Mean±SD) (n=5; *, p<0.05).

FIG. 7 depicts a synthesis scheme for HA-ester linker-rapamycin.

FIG. 8 depicts a synthesis scheme for HA-carbamate linker-rapamycin.

FIG. 9 depicts a synthesis scheme for HA-Gly-Gly-Gly-Val-Cit-Glu-Asp-rapamycin.

FIG. 10 depicts the UV/Vis absorption spectrum of an HA-Gly-Gly-Gly-Val-Cit-Glu-Asp-rapamycin conjugate.

FIG. 11 depicts gas permeation chromatograms of Na-HA (A: 201 nm; B: 280 nm) and HA-Gly-Gly-Gly-Val-Cit-Glu-Asp-rapamycin conjugates (C: 210 nm; D: 280 nm).

FIG. 12 depicts the in vitro release of rapamycin from HA-Gly-Gly-Gly-Val-Cit-Glu-Asp-rapamycin conjugates into PBS.

FIG. 13 depicts a synthesis scheme for HA-disulfide linker-rapamycin

FIG. 14 depicts a proposed pathway for release of rapamycin from an HA-disulfide linker-rapamycin.

### DETAILED DESCRIPTION OF PREFERRED EMBODIMENT

The present invention is directed to a drug conjugate comprising an mTOR inhibitor, hyaluronic acid and a linker coupling the hyaluronic acid to the mTOR inhibitor. The invention is also directed to pharmaceutical compositions comprising the drug conjugate and methods of making and using the drug conjugate.

In certain embodiments, the mTOR inhibitor is selected from the group consisting of rapamycin, everolimus, temsirolimus, deforolimus, BGT226, SF1126, BEZ235, Gedatolisib, SF1101, preferably rapamycin, everolimus, temsirolimus, and deforolimus. It was surprisingly an unexpectedly found that an mTOR inhibitor such as rapamycin could be successfully linked to hyaluronic acid, using the linkers of the present invention, to produce a stable drug conjugate. Rapamycin and other rapalogs have innate chemical instability. Well-controlled reaction conditions are necessary when working with rapamycin. Rapamycin is moisture and light sensitive, which requires reactions be performed under anhydrous conditions and protected from light if at all possible. Similar structured molecules such a rapalogs temsirolimus, everolimus and deforolimus have similar poor stability. The lactone moiety in the macrocyclic structure is sensitive to hydrolysis, which can easily disrupt its cyclic structure. This hydrolysis is base catalyzed and can readily occur in the presence of strong bases and buffer conditions above pH 9. Weak nucleophilic bases such as pyridine and DIPEA are needed to drive acyl substitution reactions of linkers and/or leaving groups to the 42-hydroxyl on rapamycin. However, using too much of a certain base will elicit rapamycin degradation, whereas too little will result in poor product yield. Amine linkers requiring fluoroenylmethoxyloxycarbonyl (Fmoc) protection can complicate reactions, as Fmoc deprotection can occur in the presence of these nucleophilic bases.

Degradation can also occur under acidic conditions. Highly acidic conditions can degrade rapamycin and rapalogs completely within minutes. This can be problematic in reactions involving oxalyl chloride and other chlorinating agents, which react violently with water to product HCl. Extra precautions must be taken to either exclude water from reactions involving these agents, or the acidic byproducts must be quenched using an appropriate amount of base. Because most reactions involving rapamycin are performed in various organic solvents, monitoring and maintaining exact pH values becomes difficult.

Rapamycin's poor water solubility and sensitivity to hydrolysis also complicates coupling to hyaluronic acid (HA). Modifications to either rapamycin or HA to improve solubility (aqueous or organic), biphasic reaction conditions, or added surfactant is needed to help alleviate the solubility gap. As such, any coupling agent used must be stable and active within these systems. Coupling agents such as HATU and PyBOP that require a base to facilitate reactions will further degrade rapamycin. Coupling agents such as DMTMM, which are ideal in weakly acidic conditions and stable in water and co-solvents such as DMSO and/or DMF may be a better solution, but the structural sensitivity of rapamycin means that typical strategies will often fail despite working with similar molecules. For example, tacrolimus, a non-rapalog, is almost structurally identical to rapamycin; however, Stak (US 20110201639 A1) teaches that tacrolimus is stable at alkaline pH conditions for over a week, whereas alkaline conditions and bases degrade rapamycin rapidly in hours or less. Procedures reported for even similar molecules are not predictive to practitioners what will be suitable for complex drugs such as rapamycin. It was therefore surprising and unexpected that rapamycin was able to be conjugated to hyaluronic acid in the drug conjugates of the present invention.

Other mTOR inhibitors are very similar in structure to rapamycin. For example, with respect to temsirolimus, everolimus, and deforolimus, the only difference is the modification of the hydroxyl group. Specifically, either of the two hydroxyl groups of rapamycin are difficult to react. The temsirolimus, everolimus, and deforolimus, are identical in structure to rapamycin, with the exception that one of the hydroxyl groups is already reacted. Thus working with temsirolimus, everolimus, and deforolimus would present similar challenges as working with rapamycin, and they would also be expected to react with the claimed linkers in a manner similar to rapamycin, such that the chemistry of adding the linkers is expected to be interchangeable. Other mTOR inhibitors, such as BGT226, SF1126, BEZ235, Gedatolisib, SF1101, would also be expected to be successfully conjugated to hyaluronic acid using the linkers of the present invention. For example, BGT226 can be conjugated using carbamate linkers. SF1126 can be conjugated using ester or carbonate linkers.

Preferred linkers for use in the drug conjugates of the present invention comprise one or more of an ester, a carbonate, or a carbamate group. The linkers preferably form a covalent biodegradable bond with the mTOR inhibitor. When the mTOR inhibitor is rapamycin or a rapalog, the bond with the mTOR inhibitor is preferably formed by an ester, carbonate, or carbamate. Such bond is preferably formed with a hydroxyl group of the mTOR inhibitor, such as the 42-hydroxyl or 31-hydroxyl group of rapamycin, the 42-hydroxyethyl group of everolimus, the 31-hydroxyl group of temsirolimus, or the 31-hydroxyl group of deforolimus.

The linker preferably forms a covalent bond with hyaluronic acid, which may be formed with the carbonyl group on the surface of the hyaluronic acid molecule. The hyaluronic acid may also be linked via the hydroxyl, carboxylic acid, or reducing sugar of HA. In certain embodiments, an amine of the linker forms an amide bond with the hyaluronic acid. The nitrogen forming the amide bond may be provided by an amino group positioned at one end of the linker. In certain embodiments, the linker comprises an amino acid that forms the amide bond with the hyaluronic acid. The linker may also form carbonyl, hydrazide, amide, urea, ester, ether, carbonate, carbamate or alkyl bonds with the hyaluronic acid.

In general, the hydroxyl group of rapalogs may be linked using an ester, carbonate, or carbamate. The linker preferably comprises at least three atoms, including any hetero atom. The linker is preferably not so long that it significantly reduces the hydrophilicity of the drug conjugate. Preferentially, an ester would be sterically hindered or bulky to reduce the susceptibility to esterases, in order to prevent cleavage of the rapamycin before it is in a tumor, cancer cell or site of preferred action. For example, a benzoic acid may be used as a hindered ester. Preferentially, the ester would contain electron donating or dense groups that would stabilize the ester bond from spontaneous hydrolysis, increasing the time required for the drug to release from the HA carrier. A benzoic acid may have electron donating groups, at para or meta or ortho positions, which further hinder the esters or donate electrons to decrease the hydrolytic cleavage rate of the ester.

Carbonates are generally less susceptible to hydrolysis and enzymatic degradation than esters. Carbamates are generally less susceptible to hydrolysis and enzymatic degradation than esters or carbonates. Cleavage of carbamates and carbonates, as well as esters, may be triggered by enzymatic, reductive or hydrolytic cleavage of another linkage on the linker, which leads to a movement of electrons to the carbonate or carbamate group linked to the rapalog. The trigger is a group that has a different rate or method of cleavage than the ester, carbamate or carbonate on the rapalog. Suitable trigger groups include biologically labile linkages such as peptide sequences and disulfide linkages. The electron transfer may involve movement of electrons via double bonds or aromatic rings. Cleavage of a trigger may lead to release of a nucleophilic group that is able to form a 5 or 6 member ring that attacks and cleaves an ester, carbamate or carbonate group, resulting in release of the rapalog.

In certain embodiments, the linker comprising a biologically labile trigger linkage is configured according to:

Rapalog-O—(C═O)V—U—X—Y-HA

Where V is an N, O, C, or an aromatic; U consists of an aromatic group, an amine, O and/or 1 or more C's; X consists of 1 or more ethylene oxides, a C, disulfide, and/or di or tri-peptide; Y consists of a carbonyl, amide, urea, ether, ester, carbonate, carbamate, alkyl, hydrazide or amine linkage to HA, where a hydroxyl, carboxylic acid, or reducing sugar of HA has been formed into a bond with linker. Other suitable linkers containing a trigger can be configured by one skilled in the art. Use of biologically labile linkage as a trigger is particularly suitable for linkers comprising carbamate and carbonate linkages with the mTOR inhibitor.

In certain embodiments, the linker is configured to stabilize the bond between the linker and the mTOR inhibitor. This could provide a sustained release that allows the drug conjugate to reach the target site before the drug is released from the conjugate. For example, the linker comprising an ester, carbonate, or carbamate may include a strong electron donating group, such as a methoxy group or other substitution on a benzene ring, that reduces the hydrolysis rate. In addition or in the alternative, the bond may be hindered by a large group on the linker, such as a benzene ring or other aromatic group, or configured in a more rigid condition. Further, structures that provide a more hydrophobic environment may limit access to the bond by serum esterase.

The linker comprising an ester preferably comprises a hindered or electron rich labile bond. When used herein, “labile” means able to be cleaved in biological systems, including in vitro models, ex vivo biological tissue or blood components, or with living organisms, such as animals or humans. In certain embodiments, the linker comprising an ester may comprise an aromatic, such as substituted or unsubstituted benzene, and/or one or more alkyl carbons. The linker comprising an ester preferably further comprises a nitrogen forming an amide bond with the hyaluronic acid. In the exemplary embodiment of Examples 1 and 2, the linker comprises 3-amino-4-methoxy-benzoate, wherein the amino group forms the amide bond with hyaluronic acid. Such a drug conjugate has been shown to be stable and exhibit sustained release and activity. Other suitable esters can be determined by one of ordinary skill of the art.

Carbamate linkages are relatively stable in plasma. Upon degradation in vivo, carbon dioxide is the only side product besides the hyaluronic acid-linker and mTOR inhibitor. In certain embodiments the linker comprising a carbamate comprises a diamine. In the exemplary embodiment of Example 3, the linker comprises 1,4-butanediamine. Other suitable carbonate and carbamates can be determined by one of ordinary skill in the art.

In embodiments wherein the linker comprises a carbonate or a carbamate, the linker preferably comprises a biologically labile linkage, as described above. The biologically labile linkage may comprise a functional group or peptide linkage that can be modified to form a more reactive intermediate, which could react with the carbonyl linked to the rapalog hydroxyl to effect release of the mTOR inhibitor.

The biologically labile linkage, such as a biologically labile peptide sequence or biologically labile disulfide linkage, that is preferentially cleaved within target cells. A linker comprising a biologically labile linkage preferably also comprises a carbonate or carbamate conjugated to the mTOR inhibitor, although biologically labile linkages can be used with an ester conjugated to the mTOR inhibitor. Cleavage of the biologically labile group within the target cell induces spontaneous lability in the carbonate, ester, or carbamate group that allows cleavage of the bond with the mTOR inhibitor within the cell.

In embodiments in which the linker comprises a biologically labile peptide sequence, the peptide may form a carbamate, carbonate, or ester linkage with the mTOR inhibitor. An amine of the peptide may also form an amide linkage with the hyaluronic acid. In certain embodiments, the peptide sequence comprises between 2 and 11 amino acids, and preferably comprises 2 to 7 amino acids, and more preferentially comprises between 2 and 3 amino acids. The amino acids may be any type of amino acid, although in certain embodiments 1 or more non-coded amino acids, such as citrulline (Cit), are used. The peptide preferably has a net positive charge. In certain embodiments, the peptide sequence contain a 2-3 member sequence, preferably at least one sequence selected from the group consisting of Phe-Lys, Val-Lys, Ala-Lys, Phe-Phe-Lys, Ala-Phe-Lys, Gly-Phe-Lys, Ac-Phe-Lys, HCO-Phe-Lys, Val-Cit, Phe-Cit, Leu-Cit, Ile-Cit, Trp-Cit, Phe-Arg(NO₂)₂, and Phe-Arg(Ts), wherein Ac is acetyl, HCO is aldehyde, Ts is tosyl and the remaining abbreviations identify amino acids.

In one exemplary embodiment discussed in Example 4, below, the linker comprises a 7-peptide sequence containing a human liver enzyme cathepsin B-labile dipeptide (Val-Cit), having the sequence Gly-Gly-Gly-Val-Cit-Glu-Asp [SEQ. ID. NO:1]. Many human cancers have demonstrated increased levels of cathepsin B expression, including breast cancer. Cathepsin B is associated with the invasiveness of breast cancer during metastasis. This drug conjugate may present improved extracellular stability as the drug would be released specifically within the cancer cells; therefore, the systemic toxicity of rapamycin or other mTOR inhibitor may be reduced. Other suitable peptide linkers can be determined by one of ordinary skill in the art.

Drug conjugates containing a disulfide linkage have demonstrated good stability in the blood stream, whereas they may be efficiently cleaved by cellular thiols, including glutathione (GSH) and thioredoxin (Trx). GSH and Trx are commonly found in cancer cells at elevated levels. Therefore the drug conjugates comprising a disulfide linkage may be attacked by cellular thiols in cancer cells, triggering the release of the active drug.

In certain embodiments with a biologically labile disulfide bond, cleavage within the cell may result in formation of a 5 or 6 member ring that is able to induce labiality in the carbonate, ester, or carbamate. In certain embodiments, the disulfide may comprise an ethyl or propyl thiol linker that can cleave the carbonate functionality to release the drug. Other suitable disulfide linkages can be determined by one of ordinary skill in the art. The linker containing a disulfide linkage may also comprise an amino group to form an amide bond with the hyaluronic acid, which amino group may be provided by an amino acid, such alanine, although other amino acids can also provide a reactive amino group, as can be determined by one of ordinary skill in the art. As exemplified in Example 5, the linker may comprise ethyldisulfide-Ala.

It was surprising and unexpected that the innately unstable rapamycin could be successfully linked to hyaluronic acid to form the drug conjugates of the present invention. As described in more detail in the Examples below, it was found that rapamycin can be linked hyaluronic acid using linkers comprising an ester, a carbonate and a carbamate linkage with the rapamycin. It would be expected that other esters, carbonates, and carbamates could be coupled to rapamycin and hyaluronic acid under the same conditions, as discussed above. As discussed above, because of the common structures and reactive sites, it would be expected that other rapalogs, such as rapamycin, everolimus, temsirolimus, deforolimus, could be coupled to hyaluronic acid using similar linker and conditions. Other mTOR inhibitors, such as BGT226, SF1126, BEZ23 Gedatolisib, or SF1101, could also be used to form drug conjugates consistent with the present invention.

Hyaluronic acid is a highly water soluble and biodegradable polymer that is distributed throughout the human body. The hyaluronic acid polymer is a polysaccharide, of alternating D-glucuronic acid and N-acetyl D-glucosamine, found in the connective tissues of the body and cleared primarily by the lymphatic system (12 to 72 hours turnover half-life). After entering the lymphatic vessel, hyaluronic acid is transported to lymph nodes where it is catabolized by receptor-mediated endocytosis and lysosomal degradation.

Several studies have correlated increased hyaluronic acid synthesis and uptake with cancer progression and metastatic potential. Breast cancer cells are known to have greater uptake of hyaluronic acid than normal tissues, requiring hyaluronic acid for high P-glycoprotein expression, the primary contributor to multi-drug resistance.

Furthermore, invasive breast cancer cells overexpress CD44, the primary receptor for hyaluronic acid, and are dependent on high concentrations of CD44-internalized hyaluronic acid for proliferation. CD44 is a cell surface molecule involved in proliferation, differentiation and migration of cancer cells. The CD44 receptor is one of the most widely accepted cell surface markers of a variety of types of cancer cells, such as breast cancer cell lines MCF-7, MDA-mB-468 and mDA-MB-231. The expression of specific CD44 isoforms is also associated with various cancer biomarkers and tumor subtypes. Hyaluronic acid is one of the principal ligands for CD44 receptor. The CD44 receptor mediates cell-cell and cell-matrix interactions through its affinity with hyaluronic acid and the adhesion with hyaluronic acid molecules plays an important role in tumor growth and progression. Thus, drug conjugates with hyaluronic acid may be efficacious against lymphatic metastases.

Accordingly, the hyaluronic acid drug conjugates of the present invention can be directed to the lymphatic system and accumulate in lymph nodes by binding to CD44 receptors on the lymph node surface and cancer cells where the CD44 receptors are overexpressed. This allows the mTOR inhibitor in the conjugate to be delivered to the site of initial tumor spread, concentrating its effects in the lymph nodes. By having lymphatic uptake as opposed to systemic absorption, the hyaluronic acid drug conjugates provide for lower organ and systemic toxicity compared to current chemotherapy delivery technologies with naked drugs. Therefore, the interaction of CD44 and HA can be used as a potential target for cancer therapy.

The molecular weight of the hyaluronic acid can be varied and has a significant effect on uptake into the lymph system and thereby affects the lymphatic drug concentration. Hyaluronic acid of between 10 kDa and 1 MDa can be used consistent with the present invention. Suitable sizes include between about 35 kDa and about 200 kDa, and all sizes and ranges there between, including 35 kDa, 75 kDa, 150 kDa, and 200 kDa.

Accordingly, the molecular weight of HA can be optimized to about 30 to 300 kDa, more preferably about 30 kDa to about 200 kDa, still more preferably from about 35 kDa to about 100 kDa, and most preferably from about 30 kDa to about 75 kDa. These lower molecular weight HA polymers can be further refined depending on the mTOR inhibitor being loaded and the accumulation characteristics of the conjugate in the lymphatic system. For example, molecular weights of 30 kDA to 50 kDa can be advantageous as well as about 35 kDa polymers. These HA polymers are sufficiently soluble so as to be capable of transporting the mTOR inhibitor conjugated thereto into the lymphatic system. Furthermore, when incorporated into the drug conjugate of the present invention, the hyaluronic acid forms a compact nanoparticle that is much smaller in at least one dimension that a linear hyaluronic acid polymer.

The drug conjugate of the present invention may be a nanoparticle configured for preferential update into a tumor or lymph node. The conjugate can be formulated for peritumor and subcutaneous injection for preferential translocation into the lymphatic system so systemic exposure is limited. The conjugate can be from about 10 to about 30 nm to avoid capillary uptake with a neutral or negative charge to maximize rapid lymphatic uptake, preferentially about 15 to 25 nm, and most preferentially about 20 nm. There is an optimum size range for lymphatic uptake of subcutaneously injected particles: particles larger than 100 nm will remain largely confined to the site of injection, particles of about 10 to 80 nm are taken up by the lymphatics, and small particles and molecules (<20 kDa) will be absorbed by the blood capillary network into systemic circulation. Conjugates larger than 100 nm or less than 5 nm are not very practical. Preferably, the conjugates can be between about 9 and 100 nm (e.g., 10, 20, 30, 40, 50, 60, 70, or 80 nm, and all sizes and ranges therebetween), more preferably between about 15 and 50 nm, and most preferably between about 20 and 40 nm.

One of the challenges of using rapamycin as an anticancer agent in clinic is due to its lipophilic chemistry. By using the hyaluronic acid as a drug delivery carrier in the drug conjugate of the present invention, the solubility of rapamycin in water can be dramatically increased from 2.6 μg/mL to more than 10 mg/mL. As such the present technology can be used to block the mTOR pathway and regulate any of the relevant mTOR pathway signals and processes.

The drug conjugates of the present invention can be loaded with the mTOR inhibitor at high rates. In certain embodiments the drug conjugate is loaded with 1% to 50% (w/w), preferably 5% to 30% (w/w), of the mTOR inhibitor, and can be any value or range therebetween. As described in Example 1, a drug conjugate of the present invention achieved rapamycin drug loading between 1 and 5%, and can be expect to achieve loading of 10% or higher. Drug conjugates of other mTOR inhibitors, such as everolimus, temsirolimus, and deforolimus, should achieve loading of 10% to 30% or higher.

The present invention further comprises methods of administering the drug conjugates of the present invention to deliver the mTOR inhibitors, such as BGT226, SF1126, BEZ235, Gedatolisib, SF1101, rapamycin, temsirolimus, everolimus, and deforolimus, to targeted sites. For example, the drug conjugates of the present invention can be used to deliver the mTOR inhibitors into lymph nodes. The drug conjugate and method can be used for anticancer treatment, for vaccination such as cancer vaccination, as an adjuvant for use with anticancer vaccines, and for other immunological uses. The drug conjugate may also be used to inhibit the PI3K/PTEN/Akt/mTOR pathway as a possible way of killing CSCs for many types of cancers, including breast, leukemia, brain, colon, lung, and prostate. The drug conjugates of the present in invention may be used alone or in combination with other treatment regimens such as radiation, other chemotherapies, and/or other HA-targeted treatments (e.g. HA-cisplatin). The drug conjugate may be used as a neoadjuvant or adjuvant therapy. Thus the invention is also directed to any of the drug conjugates of the invention for use in a method for treating cancer, vaccinating against cancer, enhancing the activity of cancer vaccines, killing CDCs, enhancing the activity of cancer treatments, and for other immunological and adjuvant uses.

The drug conjugate and method provides for the mTOR inhibitor to be targeted to lymph nodes and released slowly form the hyaluronic acid and linker. With respect to drug targeting, the size of the conjugate can be tailored for preferential update into tumors or lymph nodes. Further, the hyaluronic acid of the drug conjugates has intrinsic-CD44-tropism, such that the drug conjugates can deliver the mTOR inhibitors for localized CD44-positive breast cancer treatment. HA is also a known ligand for RHAMM, and one skilled in the art would configure the rapalog HA conjugates to target RHAMM positive cancers, including but not limited to prostate, head and neck, breast, ovarian, colon and ovarian cancers. The drug conjugate may be uptaken by cells via endocytosis. The mTOR inhibitor can then be liberated after the conjugate enters the cells.

The usage of hyaluronic acid in the drug conjugate of the present invention could provide specific cancer targeting via CD44 interaction and the benefits associated with both active-targeting nanoparticle and polymer-drug conjugates. Such biodegradable polymeric nanoparticles with the combination of targeted delivery and controlled release manner could allow drug to be specifically delivered to cancer cells per targeting bio-recognition event and minimize systemic toxicity. A steady state cytotoxic drug concentration at the tumor site over an extended period of time can be reached by this strategy. One the other hand, the polymer-drug conjugates are designed to increase therapeutic index by drug-specific targeting of disease, tissues, reducing systemic drug exposure, and increased plasma circulation time. The drug conjugates of the present invention can be bio-activated to provide their own therapeutic efficacy to the body. Moreover, the drug conjugate delivery platform has demonstrated prolonged in vivo half-life, is less prone to enzyme degradation with less immunogenicity compared to the conventional chemotherapy. It also provides an effective and promising way for neoplastic treatment due to the changing of cellular uptake mechanisms, pharmacokinetic disposition and ultimately targeting of the drug.

The drug conjugates of the present invention may also be configured for sustained release, as discussed above. For example, linkers between the rapalog and HA may incorporate hindered or bulky groups, enzyme specific groups, or cell specific groups that would sustain release over a therapeutic period in the tumor, lymph nodes, or tissues containing pretumorous sites. Sustained release is understood by one skilled in the art to include HA and rapalog conjugates that release the rapalog lower than a physical mixture of the HA and rapalog, or more preferably slower than HA conjugated to the rapalog via an unhindered or electron deficient ester. The sustained release carrier may be configured so that the rate of release of the rapalog is faster in cancerous tissues or cancer cells compared to normal tissues, plasma, or saline mixtures. Through the use of different linkers and bonds, release half-life may be further modified to be shorter or longer as desired for the intended use.

As described in Example 1, below, it was observed that in CD44 positive MDS-MB-468 cells, the drug conjugates utilizing rapamycin conjugated to hyaluronic acid by a 3-amino-4-methoxy-benzoic acid linker, the cell viability was significantly decreased compared to free rapamycin and CD44-block controls. A rat pharmacokinetics study showed that the area-under-the-curve of the drug conjugate formulation was 2.96-fold greater than that of the free drug, and the concomitant total body clearance was 8.82-fold slower. Moreover, in immunocompetent BALB/c mice bearing CD44-positive 4T1.2neu breast cancer, the rapamycin loaded hyaluronic acid particles significantly improved animal survival, suppressed tumor growth and reduced the prevalence of lung metastasis. Example 2 provides an alternative method for making such drug conjugate that is feasible for scale-up production. It would be expected that in addition to rapamycin, other mTOR inhibitors, such as rapamycin, temsirolimus, everolimus, and deforolimus, BGT226, SF1126, BEZ235, Gedatolisib, and SF1101, particularly temsirolimus, everolimus, and deforolimus, could be prepared by similar methods and achieve sustained and controlled release of the drug while maintaining activity.

Further, as described in Examples 3-5 below, rapamycin was successfully conjugated to hyaluronic acid using a variety of linkers of the present invention. It would be expected that other mTOR inhibitors, such as rapamycin, temsirolimus, everolimus, deforolimus, BGT226 SF1126, BEZ235, Gedatolisib, and SF1101, particularly temsirolimus, everolimus, and deforolimus, could be conjugated to hyaluronic acid using similar linkers. In addition, one of ordinary skill in the art can use the teachings of the Examples, including Examples 6 and 7 which describe unsuccessful synthesis attempts, to determine how to produce drug conjugates of the present invention incorporating different linkers comprising esters, carbonates, and carbamates without undue experimentation.

The pharmaceutical composition of the present invention can be configured for administration via any medially acceptable method. In certain embodiments, the pharmaceutical composition is configured for percutaneous, intradermal, mucosal or submucosal, subcutaneous, interstitial, intrafat, peritumoral, intramuscular injection mucosa, peritumorally, inhalation, instillation, systemic, intraluminal, intravenous, intranasal or intraarticular administration.

Suitable preparations for subcutaneous administration are primarily aqueous solutions of an active ingredient in water-soluble form, for example a water-soluble salt, and furthermore suspensions of the active ingredient, such as appropriate oily injection suspensions, using suitable lipophilic solvents or vehicles, such as fatty oils, for example sesame oil, or synthetic fatty acid esters, for example ethyl oleate or triglycerides, or aqueous injection suspensions which contain viscosity-increasing substances, for example sodium carboxymethylcellulose, sorbitol and/or dextran, and, if necessary, also stabilizers.

According to the methods of the present invention, the compositions of the invention can be administered by injection by gradual infusion over time or by any other medically acceptable mode. Any medically acceptable method may be used to administer the composition to the patient. The particular mode selected will depend of course, upon factors such as the particular drug selected, the severity of the state of the subject being treated, or the dosage required for therapeutic efficacy. The methods of this invention, generally speaking, may be practiced using any mode of administration that is medically acceptable, meaning any mode that produces effective levels of the active composition without causing clinically unacceptable adverse effects.

For injection, the drug conjugates can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. Preferably, the drug conjugates can be formulated in aqueous solutions, preferably in physiologically compatible buffers such as Hanks's solution, Ringer's solution, or physiological saline buffer.

The drug conjugates can be formulated for subcutaneous administration by injection, e.g., by bolus injection or continuous infusion. Formulations for injection may be presented in unit dosage form, e.g., in ampules or in multidose containers, with an added preservative. The compositions may take such forms as suspensions, solutions, or emulsions in oily or aqueous vehicles, and may contain formulator agents such as suspending, stabilizing, and/or dispersing agents.

Sterile injectable forms of the compositions of this invention may be aqueous or a substantially aliphatic suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The following non-limiting examples describe exemplary embodiments of the present invention, as well as examples of reaction conditions that did not produce observable drug conjugate.

### Example 1

### Synthesis, Characterization and Activity of HA-ester-rapamycin

### SUMMARY

### In Vitro Drug Release Study

The release profiles of free rapamycin, HA-Temsirolimus and HA-L-Rapa were fitted using first order kinetics. The release half-lives of free rapamycin (PBS), HA-Temsirolimus (PBS), HA-L-Rapa (PBS), HA-L-Rapa (Serum) were 0.16 h, 4, 7 d and 1.5 d, respectively. The rate constants of release were 0.01567 h⁻¹, 0.07881 h¹, 0.01767 h⁻¹ and 0.00344 h⁻¹ for free rapamycin (PBS), HA-Temsirolimus (PBS), HA-L-Rapa (PBS) and HA-L-Rapa (serum), respectively.

Characterizations of HA Drug Conjugates

Particle Sizes and Zeta Potentials

Particle sizes of non-conjugated HA₃₅ₖ and HA-L-Rapa with different drug loading degrees were shown in Table 1.

TABLE 1
Particle sizes and zeta potentials of HA-L-Rapa
with different loading degree (Mean ± SD).

| | Size (nm) | Zeta Potential (mV) |
|---|---|---|
| HA₃₅ₖ | 10 ± 0.045 | −77.36 ± 14.78 |
| HA-L-Rapa 1.14% (w/w) | 9.8 ± 0.124 | −56.15 ± 8.18 |
| HA-L-Rapa 2.21% (w/w) | 9.4 ± 0.012 | −44.40 ± 8.11 |
| HA-L-Rapa 4.78% (w/w) | 10.7 ± 0.014 | −13.10 ± 3.96 |

There was no significant size difference between drug conjugated and non-conjugated HA. However, the absolute value of zeta potential decreased with increasing the drug loading degree on the HA. The negative zeta potential is expected as each HA monomer has one carboxylic group (pKa ˜4.5) and one HA₃₅ₖ has approximately 87 repeating units that yield a strong negatively charged nanoparticle at pH 7.4. By conjugating with rapamycin, a large hydrophobic molecule, carboxylic groups on HA surface were partially esterified by conjugation and some adjacent negative charged carboxylic groups were shielded by the drug; therefore, the absolute zeta potential value was decreased.

Thermal Analysis

DSC

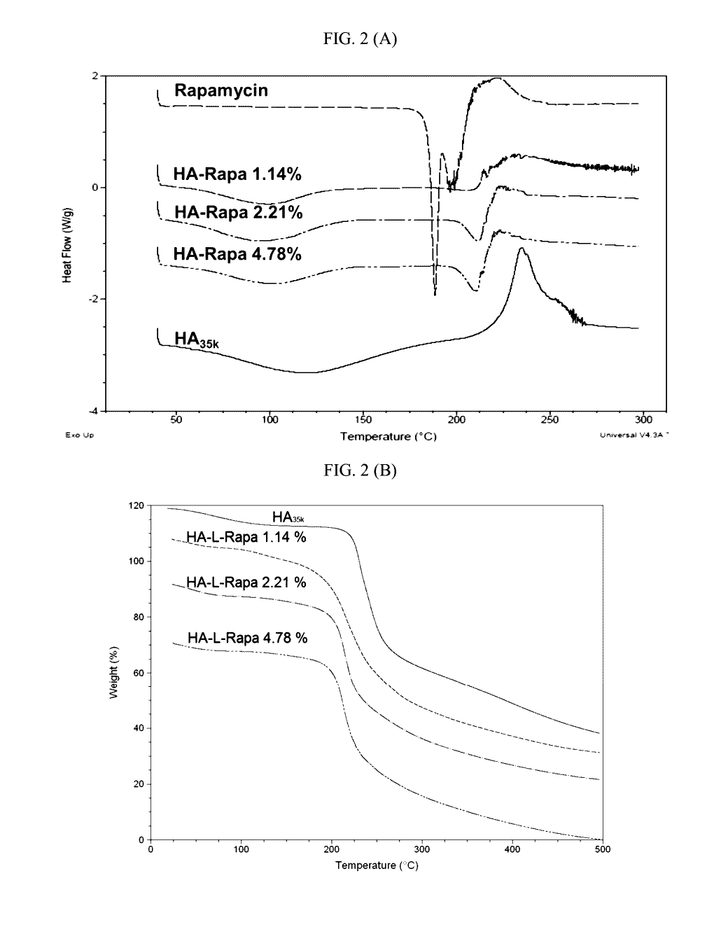

Thermally induced conformational transitions of rapamycin, HA₃₅ₖ and HA-L-Rapa with different drug loading degree are shown in FIG. 2 (A). The rapamycin showed two endothermic peaks at 187° C. and 200° C. The free HA₃₅ₖ was characterized by a broad endothermic at 120° C. and an exothermic peak at 240° C. The first broad endothermic peak of HA at approximately 120° C. suggests a dehydration process. An exothermic peak at ca. 240° C. was attributed to the decomposition of the polymer. The free rapamycin was characterized by two endothermic peaks at 187° C. and 200° C. The split peak indicated that there are two crystalline structures of pure rapamycin. However, the second exothermic characteristic peak of HA₃₅ₖ was not observed in HA-L-Rapa conjugates' DSC profiles. In addition, the two endothermic peaks of free rapamycin merged together and shifted to a higher temperature (210° C.

Both the characteristic decomposition DSC peaks of HA₃₅ₖ and rapamycin disappeared in the HA-L-Rapa profile. The exothermic peak of HA was ascribed to the melting of the polymer crystal. The disappearance of this peak indicated that the transition of HA from amorphous solid to crystalline solid was disappeared. In addition, the dehydration peak of HA shifted to a lower temperature (around 100° C.), indicating that the interaction between water and polymer decreased with conjugation of the hydrophobic rapamycin. The endothermic peak of rapamycin shifted to a higher temperature (near 210° C.). The enthalpy (AH) was calculated by integrating over the endothermic peak area. For 1.14%, 2.21% and 4.78% (w/w) drug loaded HA, the AH was 33.6±0.95, 53.58±0.83 and 65.96±2.81 J/g, respectively. This demonstrated that the degree of thermal stability of HA-L-Rapa decreased with increased drug loading degree.

TGA

Thermogravimetric analysis showed that the drug-polymer conjugates were stable up to 200° C., when degradation began [FIG. 2 (B)]. There were two transition regions during the decomposition process. For the non-drug conjugated HA, the 6.402% weight lost at approximately 100° C. was consistent with the expected water content of HA. The maximum decomposition, 72.24% wt, occurred at 235.72° C. which is consistent with polymer decomposition. The temperature of maximum degradation decreased with increasing drug loading: 224.14° C. for the 1.14% (w/w) conjugate, 218.35° C. for the 2.2% (w/w) conjugate, and 216.61 for the 4.78% (w/w) conjugate. The first transition region corresponds to the loss of water bonded to the HA molecule. The degradation temperatures of HA-L-Rapa conjugates were lower and the weight losses were smaller compared to HA₃₅ₖ. This trend is consistent with the DSC data, and it can be explained by that the water content was decreased when the hydrophobic molecules were conjugated on the HA surface. The second transition region is polymer degradation. The TGA plots illustrated that the weight loss was decreased with higher drug conjugated HA. This is also consistent with DSC data that the enthalpy at this region was increased with the drug loading degree.

Flow Cytometry

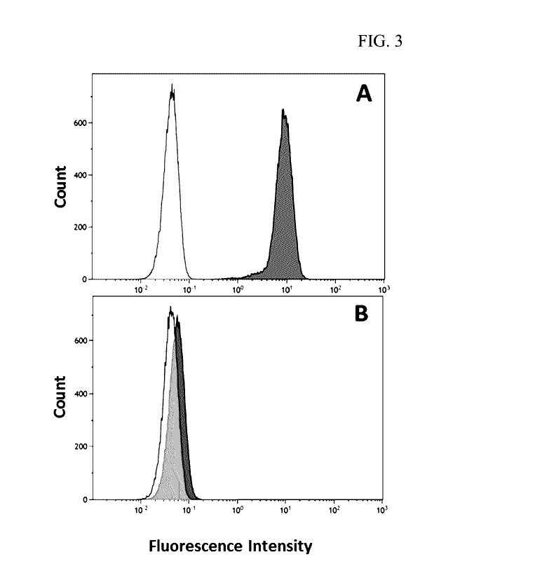

The expression of CD44 receptors on MDA-MB-468 cells was studied by flow cytometry. The cells were directly stained with PE-CD44 antibody and the PE-IgG1 isotope was used as a control (FIG. 3). Protein quantification by flow cytometry demonstrated that the percentage of CD44 positive cells in MDA-MB-468 and 4T1.2neu was 99.92% and 89.59%, respectively. When MDA-MB-468 CD44 binding sites were blocked with H-CAM, the percentage of active sites decreased to 0.57%. This result indicated that H-CAM can be used as an inhibitor to block the receptor-mediated endocytosis of HA.

In Vitro Efficacy Study

Cytotoxicity

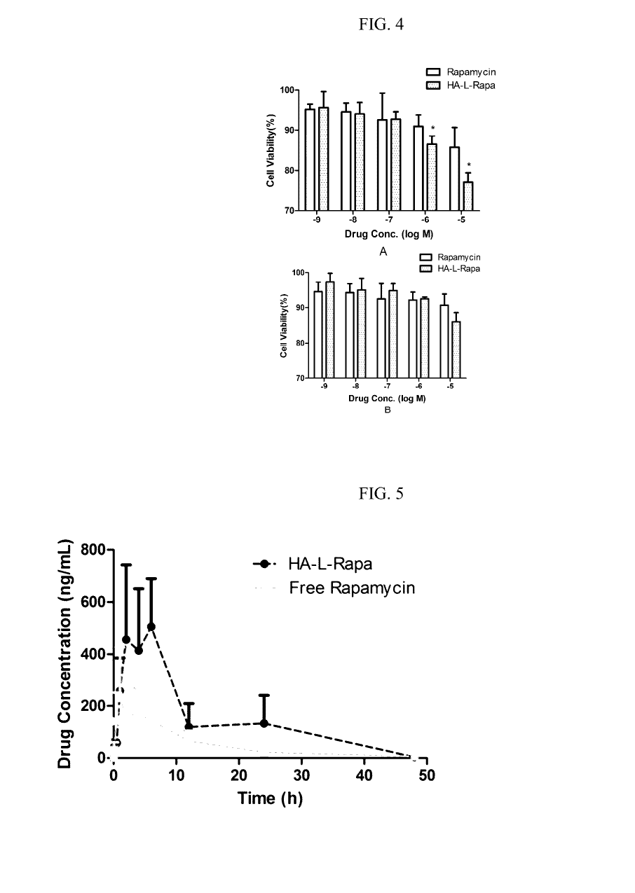

The cytotoxicity of unconjugated rapamycin and HA-L-Rapa at different concentrations was determined in MDA-MB-468 cells with or without H-CAM treatment (FIG. 4). In CD44 positive MDA-MB-468 cells, HA-L-Rapa decreased cell-viability by 8.72% compared to rapamycin (p=0.027) at 10 μM. The addition of H-CAM blocked CD44-mediated uptake and there was no significant difference in cell viability between HA-L-Rapa and the free drug (p=0.065).

Cellular Uptake Analysis

MDA-MB-468 cells with or without H-CAM in a 12-well plate were treated with free rapamycin or HA-L-Rapa at a drug concentration of 10 μM. Drug concentration in the cell culture medium was analyzed by HPLC. In CD44 positive cells, the polymer drug conjugate significantly improved the drug uptake by 3.2 times compared to the free rapamycin (p=0.012). When CD44 was blocked with H-CAM, there was no difference in rapamycin uptake between the free drug and polymer conjugate groups (p=0.13).

Pharmacokinetics Evaluation

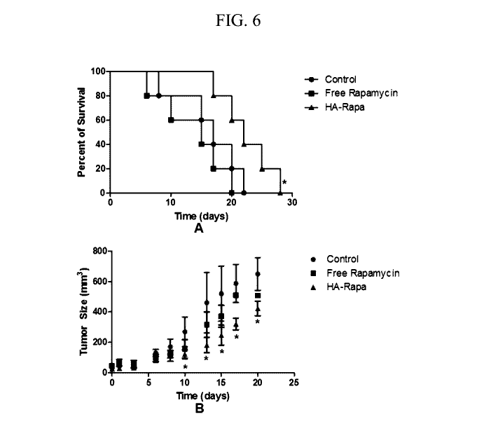

The pharmacokinetics of free rapamycin (i.p.) and HA-L-Rapa (s.c.) were compared in female Sprague-Dawley rats (n=3). A two-compartment pharmacokinetic model was selected to describe the exponential nature of the pharmacokinetics disposition of the drug (FIG. 5). The area under the plasma concentration time curve (AUC_{0→∞}) of rats administrated with HA-L-Rapa was 2.78-fold greater than that of the free drug, and the concomitant total body clearance was 2.09-fold slower, as shown in Table 2.

TABLE 2
Pharmacokinetic parameters after i.p.
free rapamycin and s.c. HA-L-Rapa.

| Parameters | Unit | Free Rapamycin i.p. | HA-L-Rapa s.c. |
|---|---|---|---|
| V_{d} | L/kg | 37.68 ± 15.49 | 12.88 ± 5.24* |
| AUC₀_{_{→}}_{∞} | (μg · h)/mL | 2.36 ± 0.46 | 6.57 ± 0.92** |
| CI | L/(kg · h) | 4.23 ± 0.95 | 2.02 ± 0.73** |
| Cₘₐₓ | ng/mL | 172.86 ± 69.06 | 544.84 ± 123.56* |
| t_{1/2} | h | 10.40 ± 3.76 | 27.95 ± 13.33* |

(Mean ± SD,
n = 3)
(*p < 0.05;
**p < 0.01)

Animal Survival and Tumor Suppression Studies

BALB/c mice were inoculated with 4T1.2neu cells to evaluate the attenuation effect of HA-L-Rapa on overall tumor progression. The median survival times of control, free rapamycin and HA-L-Rapa treatment groups were 17, 15 and 22 days, respectively (FIG. 6 A). Regression analysis demonstrated that HA-L-Rapa treatment was associated with significantly longer survival of mice with mouse mammary carcinoma compared with both the untreated control group (p=0.047) and free drug treatment group (p=0.018).

The in vivo 4T1.2neu breast cancer model also illustrated a significant decrease (p=0.049) in tumor volume on day 20 in BALB/c mice treated with HA-L-Rapa (10 mg/kg equivalent rapamycin) compared with that of the control group. Free rapamycin (10 mg/kg) also decreased tumor volume; however, the difference was not significant (p=0.056).

Tissue Distribution

Twelve hours after s.c injection of HA-L-Rapa, the drug concentrations in tumor, lymph and lung were 1.56, 2.78 and 3.23-fold greater than the free drug treatment group (Table 3). The order of drug concentrations for the control group, free rapamycin (i.p.), were tumor>lymph>lungs>kidneys>heart>liver>muscle>spleen>brain. The drug concentration of HA-L-Rapa (s.c.) formulation were lungs>lymph>tumor>spleen>muscle>liver>kidneys>heart>brain.

TABLE 3
Mean concentration of rapamycin in mice tissues measured at
12 h post administration of 10 mg/kg equivalent rapamycin
by i.p. (free rapamycin) and s.c. (HA-L-Rapa) injection.

| Tissue | Free Rapamycin (μg/g) | HA-L-Rapa (μg/g) |
|---|---|---|
| Brain | 107.69 ± 37.25 | 195.01 ± 48.56* |
| Kidneys | 469 ± 27.27 | 495.04 ± 349.05 |
| Tumor | 1237.01 ± 256.20 | 1931.84 ± 195.46** |
| Lymph | 705.28 ± 115.87 | 1972.78 ± 634.80** |
| Heart | 440.73 ± 81.57 | 358.88 ± 110.31 |
| Liver | 378.72 ± 40.56 | 565.56 ± 54.06* |
| Lungs | 617.53 ± 298.54 | 1990.00 ± 634.80** |
| Muscle | 315.56 ± 154.12 | 927.64 ± 453.48* |
| Spleen | 183.01 ± 66.46 | 1386.31 ± 342.71** |

(Mean ± SD,
n = 5)
(*p < 0.05;
**p < 0.01)

Discussion

In this study, the use of HA as a drug delivery carrier that can enhance the efficacy of the conjugated rapamycin against CD44 positive cancer cells was described. Previously our lab showed that the t_{50%} of the lymphatic drainage of medium length HA (35 kDa-74 kDa) to the axillary lymph node was 15-17 h and the tₘₐₓ was around 2 h. The release half-life of rapamycin from HA-Temsirolimus in PBS was approximately 4 h. The bulk of the drug would therefore be released before the polymer cleared from the target site. However, the sustained release characteristics can be improved by using 3-amino-4-methoxy-benzoic acid instead of ADH as a linker to conjugate the drug. This can be explained as HA-Temsirolimus was prepared using an unhindered ester, which allows rapid hydrolysis and release of the drug in water and serum. In comparison, the ester bond in HA-L-Rapa is stabilized by the para site methoxy group on the benzene ring that served as a strong electron donating group and reduced the hydrolysis rate. In addition, the ester bond in HA-L-Rapa was more hindered and the drug was in a more rigid condition. These structural configurations provided a more hydrophobic environment than that of HA-Temsirolimus, which may limit access by serum esterase. The release half-life was increased to approximately 36 h in serum supplemented PBS. This could provide a sustained release of the drug at the targeted tissue and minimize the systemic toxicity by reducing the necessary drug dose and limiting drug non-targeted tissue exposure.

Low molecular weight HA (less than 10 kDa) was reported to reversibly bind CD44 and is associated with immunogenicity. However, higher molecular weight HA (greater than 30 kDa) binds irreversible to CD44 due to the increased multivalent interactions. The HA₃₅ₖ used in this study has approximately 87 D-glucuronic acid repeating units. The 2.6% w/w loading is equivalent to one rapamycin per one polymer chain, so over 98% of the glucuronic side chains are available for binding CD44.

The in vitro results of the antibody blocking studies showed that the internalization of HA-drug conjugate was inhibited by the H-CAM CD44 inhibitor, which blocked endocytosis and CD44 specific uptake. Since HA-L-Rapa entered the cells through an endocytic pathway, inhibition of this pathway resulted in a reduction of the internalization degree of the polymer drug conjugate. Cellular uptake of the lipid permeable free drug is driven by a concentration gradient. After equilibrium is established, no more drug is able to enter cells, hence inhibition of CD44 receptor did affect free rapamycin uptake by MDA-MB-468 cells. Receptor mediated transport of HA-L-Rapa improved drug delivery in CD44 positive cells and the cytotoxicity was also significantly enhanced. These results also indicated that conjugate of rapamycin does not inhibit the HA-CD44 interaction at the amounts studied; this strategy could be utilized as a novel drug delivery platform for targeted chemotherapy with rapamycin.

This study limited rapamycin to i.p. injection and the conjugate to local s.c. administration. Rapamycin is poorly water soluble and no safe i.v. formulation has been reported. Clinical trials of i.v. rapamycin resulted in injury (swelling and focal lesion) at the injection site, lymphoid atrophy and periarterial edema in the heart, liver (FDA NDA 21-083). Our own previous rat studies demonstrated significant morbidity and a 40% mortality of i.v. rapamycin in rats. Rapamycin cannot be given subcutaneously repeatedly and safely as the free drug. Myckatyn reported skin ulceration in mice administrated 2 mg/kg rapamycin (one fifth of our dose), and given the ulceration potential of the 4T1.2neu model, this control study was not permitted by institutional animal care guidelines. Therefore, in this study, intravenous administration of rapamycin was not investigated.

The pharmacokinetics profile of s.c. HA-L-Rapa was greatly altered compared to the standard i.p. rapamycin formulation. The high value of V_{d} of free rapamycin results from its lipophilicity and thus high tissue distribution. The HA conjugate significantly reduced the volume distribution possibly by minimizing nonspecific tissue binding. The increased AUC and slower clearance rate of s.c. HA-L-Rapa are consistent with the sustained release of the drug from the conjugate.

HA targets to CD44 receptors and could specifically bind to CD44 positive cells. HA molecules are uptake by the cells through CD44 receptor-mediated endocytosis followed by lysosomal degradation. The distribution of rapamycin in the HA-L-Rapa treated mice was mainly in the tumor. The significant improvement in exposure drug in target tissues by HA-L-Rapa suggested that a lower dose of rapamycin may achieve a therapeutic effect.

In addition, the 4T1 is a highly metastatic cancer cell line. At necropsy, lung metastases were observed in 5/5 of the free rapamycin group and only 1/5 of the HA-L-Rapa group. This is consistent with that more drugs were detected in HA-L-Rapa treated animals' lungs. The lung accumulation can be explained by the prevalence of four major HA-binding proteins that potentially contributed to lung pathology regulation: CD44, toll-like receptor (TLR4), HA-binding protein 2 (HABP2) and receptor for HA-mediated motility (RHAMM) (31). Meanwhile, HA constitutes the major glycosaminoglycan in lung tissue and it has diverse function in lung homeostasis and pulmonary disease.

HA is cleared from tissues mainly by the lymphatic system due to the presence of lymphatic endothelial hyaluronan receptor, LYVE-1. The expression of LYVE-1 is largely restricted to lymphatic vessels and splenic sinusoidal endothelia cells. The LYVE-1 receptor has a 41% homology to the HA-binding CD44 receptor. This provided an additional HA targeting mechanism. It is consistent with HA-L-Rapa treated mice, where more drug accumulated in the lymph node and the spleen compared to the free drug treatment group.

The breast cancer cell line, 4T1, has an inherent propensity of ulceration. Our data illustrated that HA-L-Rapa treatment significantly inhibited tumor growth and diminished the incidence of ulcerated tumor in mammary carcinoma bearing mice. The free rapamycin treatment group showed smaller tumor sizes compared to the non-treatment group. However, the animals were sacrificed due to the presence of hemorrhagic skin ulcers and there was no statically significant survival benefit compared to saline.

Rapamycin is a promising therapeutic agent with both immunosuppressant (mTOR inhibitor) and anti-tumor activities. The immunosuppressant effect of rapamycin comes from the inhibition of T and B cell proliferation, which is the same mechanism of anticancer activity. However, based on currently available evidence, the anti-neoplastic activity is more dominant than that of immunosuppressant effects. In our study, we developed a formulation that can target the drug specifically to the tumor and lymphatic tissue via a CD44 interaction. This could further minimize the systemic immunosuppressant activity of rapamycin and augment the anti-cancer effects of the drug.

These results suggest that the rapamycin loaded HA nanoparticle could be used as a potential therapeutic agent for CD44 positive cancers.

Experimental

Materials

HA₃₅ₖ and rapamycin were purchased from Lifecore Biomedical, Inc. (Chaska, Minn.) and LC Laboratories (Woburn, Mass.), respectively. Fmoc-3-amino-4-methoxy-benzoic acid was purchased from AnaSpec, Inc. (Fremont, Calif.). Other materials and solvents, of their highest grade, were purchased from Fisher Scientific (Lenexa, Kans.) or Sigma Aldrich (St. Louis, Mo.).

### Synthesis of rapamycin 42-hemisuccinate

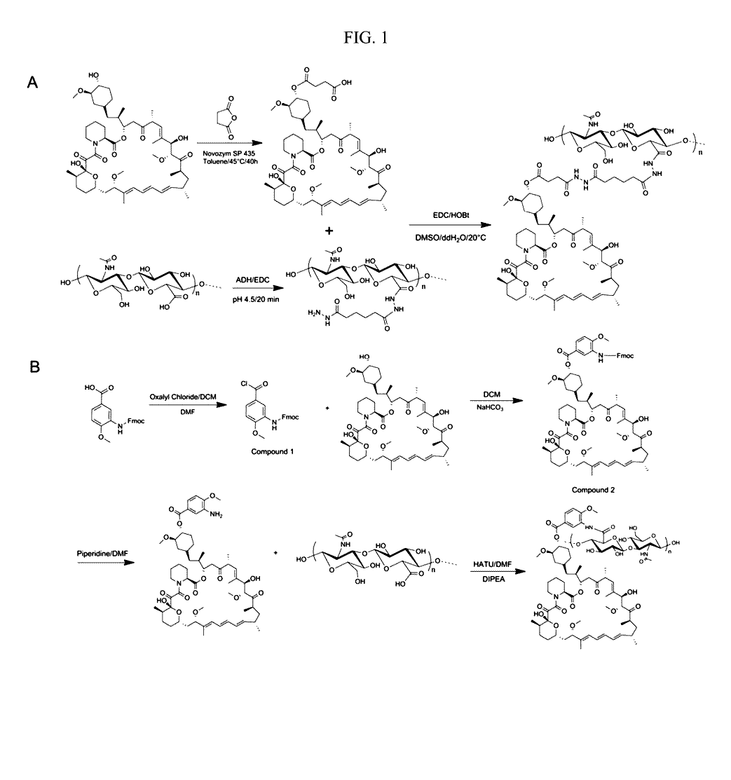

The synthetic scheme is shown in FIG. 1 A. A mixture of rapamycin (0.20 g, 0.22 mmol), succinic anhydride (0.10 g, 1.0 mmol) and Novozym SP 435 (0.45 g) in toluene (10 mL) was stirred at 45° C. under argon for 40 h. The enzyme was filtered off and washed with toluene, and the combined organic phases were concentrated under reduced pressure. The residue was purified by silica gel column chromatography and eluted with EtOAc-hexane (1:4) to furnish the title compound as a white solid (0.2 g, 90%).

### Synthesis of HA-Temsirolimus

The 42-hemisuccinated rapamycin (0.150 g, 0.15 mmol) was dissolved in 4 mL of dimethyl sulfoxide (DMSO), and 1-ethyl-3-(3-dimethylaminopropyl) carbodiimide hydrochloride (EDC.HCl) (0.057 g, 0.30 mmol) and 1-hydrocybenzotriazole hydrate (HOBt.H₂O) (0.046 g, 0.30 mmol) were added to the solution. After 20 min, this solution was added drop wise to HA-adipic acid dihydrazide (ADH) (0.120 g, 22% ADH) in 10 mL of double distilled water (ddH₂O) cooled on ice. HA-ADH was synthesized as previously described. After the addition, the mixture was stirred at ambient temperature (ca. 20° C.) overnight. Then, the solution was poured into 100 mL of 95% ethanol (EtOH), and the white precipitate was collected by centrifugation. This procedure was repeated another two times. The collected solid was dried under vacuum overnight and 0.085 g of the product was obtained (yield: 31.48%). The structure was verified by ¹H-NMR (supplementary data).

### Synthesis of HA-Rapamycin-42-(3′-amino-4′-methoxy)benzoate (HA-L-Rapamycin)

The synthetic scheme of HA-L-rapamycin is shown in FIG. 1 B.

Five milliliters of oxalyl chloride in dry methylene chloride (2.0 M in DCM) was added to 150 mg of Fmoc-3-amino-4-methoxy-benzoic acid along with one drop of dry dimethylformamide (DMF) as a catalyst. The mixture was stirred at ambient temperature (ca. 22° C.) under dry argon for 2 h. The white suspension turned into a light yellow, clear solution as the reaction neared completion. The organic solvent was removed under reduced pressure.

Compound 1 was suspended in 5 mL of dry DCM and 100 mg of rapamycin and 200 mg of NaHCO₃ were added to the solution. The mixture was stirred at ambient temperature for 2 h under dry argon and protected from light. The suspension was filtered, and the filtrate was washed with bicarbonate water and brine. The organic solvent was dried with Na₂SO₄ and then removed under reduced pressure.

Compound 2 was suspended in 20% (v/v) piperidine in DMF. The solution was stirred at ambient temperature for 1 h. The organic solvent was removed under reduced pressure and the pale yellow solid was washed several times with ddH₂O.

O-(7-azabenzotriazol-1-yl)-N,N,N′,N′-tetramethyluronium hexafluorophosphate (HATU) (100 mg, 0.26 mmol) in 1 mL of DMF and N,N-Diisopropylethylamine (DIPEA) (47 μL, 0.27 mmol) were added to 100 mg HA in 5 mL of ddH₂O. After 30 min, rapamycin-42-(3′-amino-4′-methoxy) benzoate in 5 mL of DMF was added. The mixture was stirred at ambient temperature overnight. Then, the organic solvent was removed by dialysis (10 k MWCO snake skin pleated dialysis tubing, Thermo Scientific, Rockford, Ill.) against ddH₂O. The unbound drug produced a light yellow precipitate that was removed by centrifugation. The drug conjugate was further purified by tangential flow filtration (TFF) using a MicroKros mPEG filter (3.5 k MWCO, Spectrum Labs, Rancho Dominguez, Calif.). The rapamycin loading degree on HA was determined by ¹H-NMR (supplementary data).

In Vitro Drug Release Study

The in vitro release of the rapamycin from HA-drug conjugates into PBS or PBS supplemented with 10% fetal bovine serum (FBS) at 37° C. was monitored by a dialysis method using a SnakeSkin® pleated dialysis tubing (3,500 MWCO) (24). To prevent bacteria growth, 0.05% sodium azide was added to the serum release medium, and the medium was changed several times a day to maintain sink conditions. After the predetermined time intervals, samples were withdrawn from the dialysis tubing and analyzed using a Spectra MaxM2 microplate spectrophotometer with UV detection of rapamycin at 260 nm.

HA-Rapamycin Conjugate Characterization

Particle Size. HA and HA-L-Rapa with different loading degrees were dissolved in PBS at the concentration of 2.5 mg/mL. The particle sizes were measured with a ZetaPALS (Brookhaven Instruments Corp.) using the intensity weighted Gaussian distribution.

Zeta Potential.

HA and HA-L-Rapa with different loading degree were dissolved in 10 mM KCl at the concentration of 2.5 mg/mL. Zeta potentials were measured using ZetaPALS. Zeta potential was calculated from the mobility of the system fitted into the Smoluchowski model.

Thermal Analysis.

Differential scanning calorimetry (DSC) of HA-L-Rapa with different loading degrees and HA₃₅ₖ polymer, as received, were studied using a Q100 Universal V4.3A DSC (TA Instruments, New Castle, Del.). The samples were sealed in a standard aluminum pan and heated from 40 to 300° C. at a scan rate of 10° C./min.

Thermo gravimetric analysis (TGA) was performed on a Q50 thermogravimetric analyzer from TA Instruments. Samples were loaded on a platinum sample pan and heated from 25 to 300° C. with a heating rate of 10° C./min. Data were analyzed using Universal Analysis 2000 (version 4.3A) software (TA Instrument).

Flow Cytometry Analysis

The expression of CD44 receptor on the surface of breast cancer cells, MDA-MB-468 and 4T1.2neu, was examined by flow cytometry analysis. PE mouse anti-human CD44 (BD Pharmingen, San Jose, Calif.) was used to stain MDA-MB-468 cells, and PE rat anti-mouse CD44 (Pgp-1-R-PE, Southern Biotech, Birmingham, Ala.) was used with murine 4T1.2neu cells. PE mouse IgGI isotope control (BD Pharmingen, San Jose, Calif.) was used as a control. Anti-human CD44 antibody (H-CAM, Thermo Scientific, Rockford, Ill.) was used in receptor blocking assays.

Cytotoxicity Assay

Breast cancer MDA-MB-468 cells were maintained in Dulbecco's modified eagle medium supplemented with 10% fetal bovine serum (Hyclone Laboratory Inc., Logan, Utah). Cells were plated in white 96-well flat-bottomed plates at the concentration of 5,000 cells/well in 90 μL of growth medium. After 12 h, rapamycin or HA-L-Rapa in Hanks' solution were added at different concentrations. Hanks' solution and 10% trichloroacetic acid (TCA) were used as negative and positive control, respectively. The medium was refreshed 8 h after treatment. After 72 h post-treatment, resazurin blue (5 μM) was added and the resorufin product was measured with a fluorophotometer using an excitation wavelength of 550 nm and an emission wavelength of 590 nm.

Cellular Uptake Study

Breast cancer cells, MDA-MB-468, were seeded in a 12-well plate at the concentration of 50,000 cells/well in 1 mL of growth medium. After 12 h incubation, 10 μL of human anti-CD44 antibody was added to each well. After 1 h, 10 μM of rapamycin or HA-L-Rapa conjugate were added to the cells. The supernatant was then analyzed by HPLC with a reverse phase column (TSK-GEL® ODS-100Z, Tosoh Bioscience) at 50° C. and UV detection at 278 nm for rapamycin.

Pharmacokinetics Study

Female Sprague-Dawley rats (350-450 g, Charles Rivers) were administered rapamycin (1 mg/mL in formulation buffer) by intraperitoneal (i.p.) injection or HA-L-Rapa (10 mg/kg equivalent rapamycin; n=3 for each group) by subcutaneous (s.c) injection under isoflurane anesthesia. Whole blood was withdrawn (100 μL) from the tail vein at 0 min, 5 min, 30 min, 1 h, 2 h, 4 h, 6 h, 12 h, 24 h and 48 h after dosing and placed in heparinized tubes (BD Vacutainer® Lithium Heparin 37 USP unit, BD Franklin Lakes, N.J.). The whole blood was centrifuged at 15,000×g for 10 min, and the plasma was frozen at −80° C. until analyzed. The animal use statement was approved by the University of Kansas Institutional Animal Care and Use Committee. Plasma samples and stander curves were prepared using a procedure reported previously.

Animal Survival Study and Tissue Distribution

The murine breast cancer 4T1.2neu cell line was used to establish the synergetic orthotropic tumor model in immunocompetent mice. Female BALB/c mice (20-25 g, Charles Rivers) under isoflurane anesthesia were inoculated in the right mammary gland with 1×10⁶ cells suspended in PBS. Treatment started when the tumor size reached 50 mm³. Free rapamycin was dissolved in anhydrous ethanol and reconstituted in formulation buffer before use. The formulation buffer of free rapamycin consisted of 5% polyethylene glycol 400 and 5% Tween 80 in Hanks' balance salt solution. HA-L-Rapa was dissolved in Hanks' solution. Mice received 10 mg/kg equivalent rapamycin once per week for 3 weeks by i.p. injection (free rapamycin) or s.c injection (HA-L-Rapa). Control animals were injected with Hanks' solution. Animals were sacrificed when tumors grow larger than 1000 mm³ or if the tumors ulcerated in accordance with the approved animal use protocol.

Drug tissue distribution was determined in female BALB/c mice (n=5). Tissue samples (50 mg) in 500 μL PBS were homogenized using a Tissue Tearor (BioSpec Products, Inc., Bartivesville, Okla.). The homogenized tissue was mixed with 250 μL ZnSO₄ and 500 μL methanol. The mixture solution was centrifuged and the supernatant was analyzed by LC/MS.

In the tissue distribution study, BLAB/c mice were administered the drugs 12 h before being euthanized. Major organs (liver, kidneys, hear, spleen, lungs, brain, muscle), tumor and lymph nodes were excised and lightly washed with PBS. The organs were stored at −80° C. until analyzed by LC/MS.

Statistical Analysis

GraphPad Prism 5 software was used for statistical analysis. A t-test was used for statistical analysis of comparing two means. The Mantel-Cox test was used for comparison of Kaplan-Meyer analysis. In all comparisons, statistical significance was set at p≦0.05.

### Example 2

### Alternate Synthesis of HA-ester-rapamycin

### Synthesis of N-Fmoc 3-amino-4-methoxy-benzoyl chloride

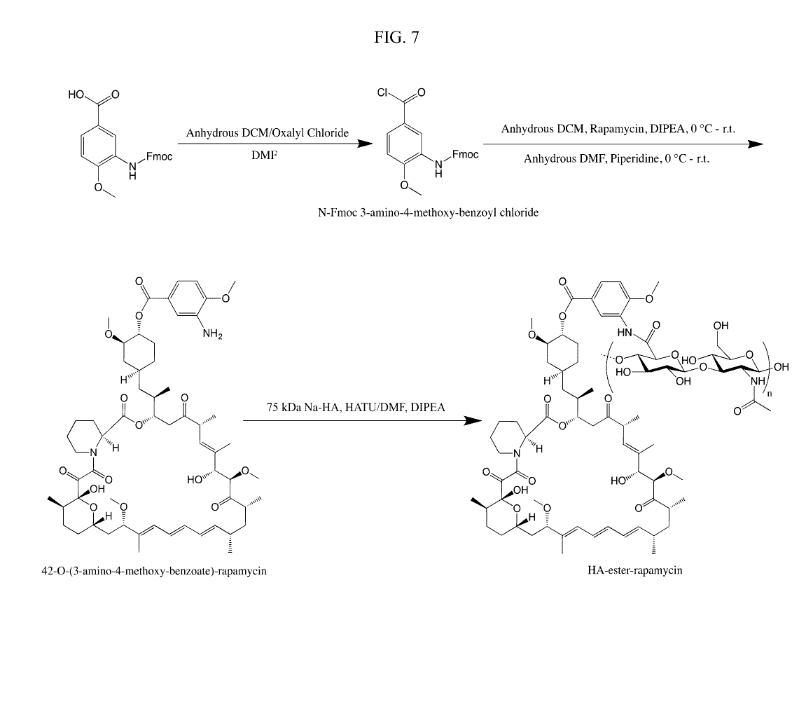

N-Fmoc 3-amino-4-methoxy-benzoic acid (150 mg, 0.39 mmol) was dissolved into 24.6 mL anhydrous dichloromethane (DCM). To this solution, oxalyl chloride (5 mL, 58.3 mmol) was added, followed by the addition of one drop of anhydrous DMF as a catalyst. The solution was stirred at room temperature for 2 hours under argon. The solution went from a cloudy white suspension to a clear yellow solution as chlorination proceeded. Excess DCM and oxalyl chloride were removed under reduced pressure overnight to afford a yellow solid. The synthetic scheme is shown in FIG. 7. MS (ESI) calculated for C₂₃H₁₈ClNO₄ (M+H)⁺: 408.09. found 407.15.

### Synthesis of 42-O-(3-amino-4-methoxy-benzoate)-rapamycin

Rapamycin (100 mg, 0.11 mmol) was dissolved in 3 mL of anhydrous DCM and cooled to 0° C. Hünig's base (38.3 μL, 0.22 mmol) was added to the solution and the reaction mixture was stirred for 15 minutes at 0° C. To this solution N-Fmoc 3-amino-4-methoxy-benzoyl chloride (90 mg, 0.22 mmol) is dissolved into 3 mL of anhydrous DCM dropwise. The reaction was allowed to warm to room temperature and proceeded overnight, after which the solvent was removed under reduced pressure. Without further purification, the solid was dissolved in 4-mL of DMF, followed by the addition of 1-mL of piperidine at 0° C. The solution was stirred at 0° C. for 30 minutes and room temperature for 1 hour. Solvent was removed under reduced pressure, and the solid was washed several times with DCM and subsequently with ddH₂O. The synthetic scheme is shown in FIG. 7.

### Synthesis of HA-ester-rapamycin

One hundred milligrams of sodium hyaluronate (Na-HA, 75 kDa, 0.25 mmol) was dissolved in 5 mL of ddH₂O. To this solution, O-(7-azabenzotriazol-1-yl)-N,N,N′,N′-tetramethyluronium hexafluorophosphate (HATU) (100 mg, 0.26 mmol) and Hünig's base (47 μL, 0.27 mmol) are dissolved in 1 mL of DMF dropwise. After 30 minutes, 42-O-(3-amino-4-methoxy-benzoate)-rapamycin dissolved in 5 mL of DMF was added slowly. The mixture was stirred at room temperature for 24 hours. Organic solvent was removed via dialysis (10 k MWCO snake skin dialysis tubing) against ddH₂O. Drug conjugate was further purified using tangential flow filtration (TFF). The synthetic scheme is shown in FIG. 7.

### Example 3

### Synthesis of HA-carbamate-rapamycin

### Synthesis of 42-O-(4-nitro-phenyloxycarbonyl)-rapamycin

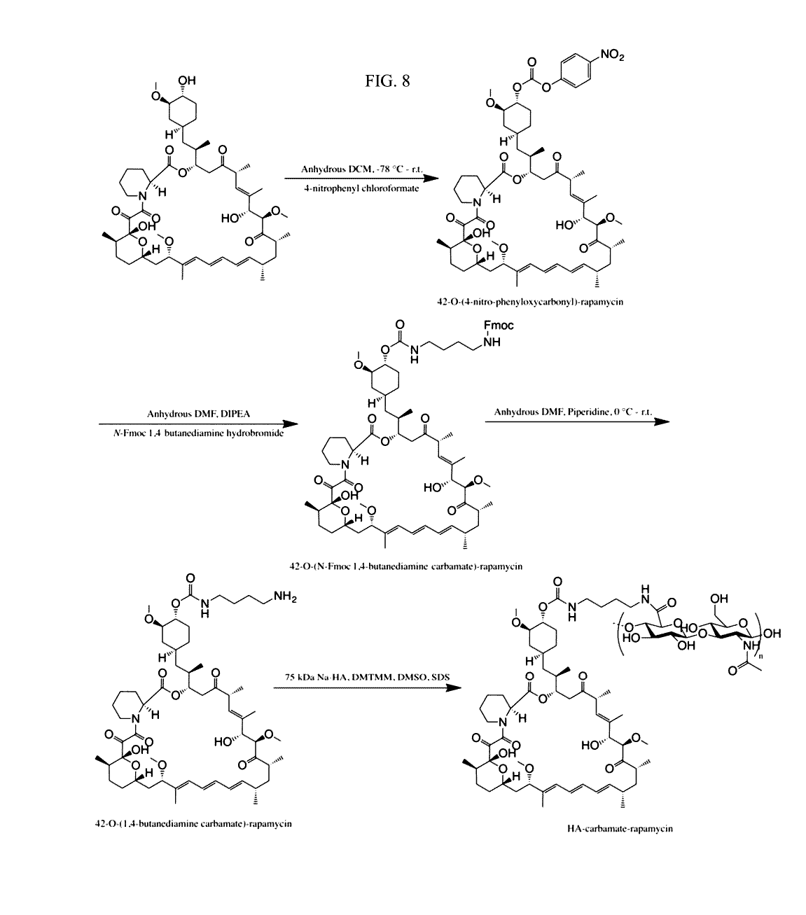

Rapamycin (200 mg, 0.22 mmol) was dissolved in 1.5-mL of anhydrous dichloromethane (DCM) and cooled to −78° C. under argon. To this solution anhydrous pyridine (213 μL, 2.64 mmol) was added dropwise and stirred for 15 minutes. 4-nitrophenyl chloroformate (44.3 mg, 0.22 mmol) dissolved in 1-mL of anhydrous DCM was added dropwise. The solution was reacted at −78° C. under argon and protected from light for 1 hour, then allowed to warm to room temperature (r.t.) and stirred for 1 hour. The mixture was diluted using DCM then washed twice with a 0.1N HCl solution and once with brine. The organic layer was dried over NaSO₄ and filtered, and solvent was removed under reduced pressure to afford a pale yellowish solid. 42-O-(4-nitro-phenyloxycarbonyl)-rapamycin was purified over silica gel with 30% ethyl acetate in hexane. The synthesis scheme is shown in FIG. 8. MS (ESI), calculated for C₅₈H₈₂N₂O₁₇ (M+Na)⁺: 1101.55. found 1101.55.

### Synthesis of 42-O-(1,4-butanediamine carbamate)-rapamycin

42-O-(4-nitro-phenyloxycarbonyl)-rapamycin (100 mg, 0.093 mmol) was dissolved in 8 mL of anhydrous DMF. To this solution N-Fmoc 1,4-butanediamine hydrobromide (73.4 mg, 0.188 mmol) was added. Hünig's base (65.3 μL, 0.375 mmol) was added dropwise, and the reaction mixture was stirred under argon protected from light for 12 hours, after which solvent was removed using reduced pressure. MS (ESI), calculated for C₇₁H₉₉N₃O₁₆ (M+Na)+: 1272.70. found 1272.90. Without further purification, the solid was dissolved in 4-mL DMF, followed by the addition of 1-mL piperidine at 0° C. The solution was stirred at 0° C. for 30 minutes and r.t. for 1 hour. Solvent was removed under reduced pressure, and the solid was washed several times with DCM and subsequently with ddH₂O. The synthesis scheme is shown in FIG. 8.

### Synthesis of HA-carbamate-rapamycin

Twenty milligrams of sodium hyaluronate (Na-HA, 75 kDa, 0.05 mmol) and DMTMM (27.5 mg. 0.1 mmm01) were dissolved in 1-mL of ddH2O with 0.5 wt % sodium dodecyl sulfate (SDS). After the mixture was stirred at r.t. for 20 minutes, a solution of 42-O-(1,4-butanediamine carbamate)-rapamycin (10.3 mg. 0.01 mmol) in 2-mL DMSO with 0.5 wt % SDS was added dropwise. The reaction was stirred in the dark at r.t. for two days. The organic solvent was removed by dialysis (10 kDa MWCO dialysis tubing) against ddH2O for 24 hours. The product, HA-carbamate-rapamycin conjugates, were further purified by washing several times with ddH2O in a 20-mL centrifugal filter (PES, 10 kDa MWCO), and finally freeze-dried. The synthesis scheme is shown in FIG. 8.

### Example 4

### Synthesis and Characterization of HA-Gly-Gly-Gly-Val-Cit-Glu-Asp-rapamycin

### Synthesis of 42-O-(4-nitro-phenyloxycarbonyl)-rapamycin

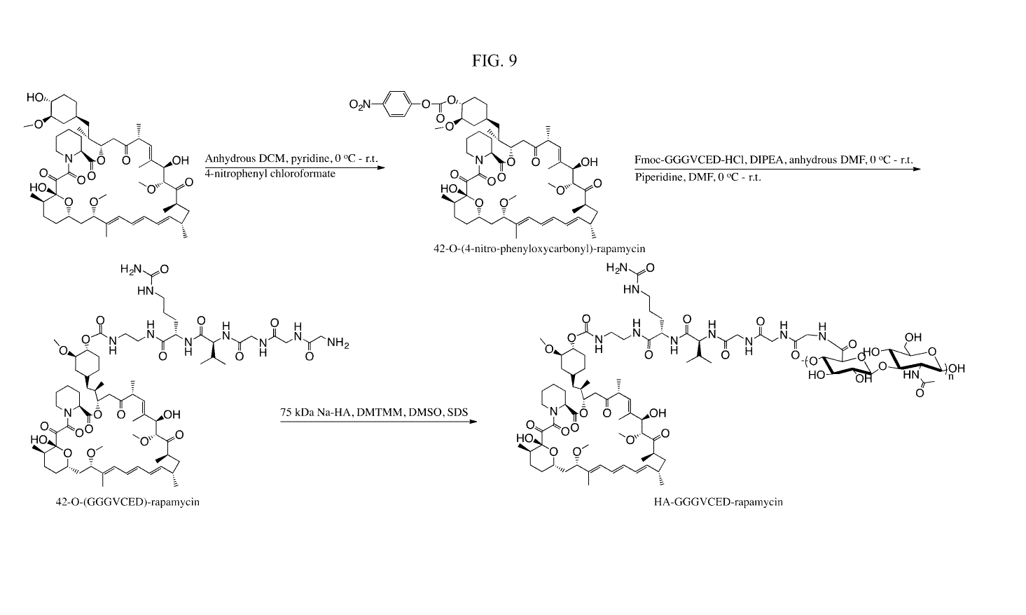

Rapamycin (200 mg, 0.22 mmol) was dissolved in 5-mL anhydrous dichloromethane (DCM) and cooled to 0° C. To this solution, pyridine (200 μL, 2.5 mmol) was added, followed by the addition of a solution of 4-nitrophenyl chloroformate (88 mg, 0.44 mmol) in 1-mL DCM. The solution was allowed to warm to room temperature (r.t.) overnight and was stirred at r.t. for 24 hours under argon in the dark. The mixture was diluted using DCM then washed three times with water and once with brine. The organic layer was dried over NaSO₄ and filtered, and solvent was removed under reduced pressure to afford a pale yellowish solid. The reaction scheme is shown in FIG. 9. Chromatography over silica gel with 30% ethyl acetate in hexane showed 160 mg of the 42-O-(4-nitro-phenyloxycarbonyl)-rapamycin as a white solid with a yield of 68%. MS (ESI), calculated for C₅₈H₈₂N₂O₁₇ (M+Na)⁺: 1101.55. found 1101.55. ¹H-NMR (400 MHz, CDCl₃) δ (ppm): 8.3 (d, J=8 Hz, 2H), 7.42 (d, J=8 Hz, 2H), 6.44-6.30 (m, 2H), 6.20-6.14 (m, 1H), 5.99 (d, J=8 Hz, 1H), 5.59-5.53 (q, 1H), 5.44 (d, J=8 Hz, 1H), 5.30 (d, 1H), 5.25-5.12 (m, 1H), 4.64 (m, 1H), 4.20 (d, J=4 Hz, 1H), 3.89 (m, 1H), 3.75 (d, J=4 Hz, 1H), 3.72-3.66 (m, 1H), 3.63-3.55 (m, 1H), 3.51 (m, 2H), 3.47 (s, 3H), 3.36 (m, 3H), 3.14 (s, 3H), 2.82-2.57 (m, 4H), 2.37-2.35 (m, 2H), 2.27-2.14 (m, 3H), 2.04-1.95 (m, 3H), 1.8-0.8 (m, 45H).

### Synthesis of 42-O-(Gly-Gly-Gly-Val-Cit-Glu-Asp)-rapamycin

42-O-(4-nitro-phenyloxycarbonyl)-rapamycin (160 mg, 0.15 mmol) was completely dissolved in 8-mL anhydrous DMF and cooled to 0° C. in an ice bath. To this solution was added a solution of Fmoc-Gly-Gly-Gly-Val-Cit-ethylenediamine, HCl salt (133 mg, 0.18 mmol), and Hünig's base (200 μL, 1.15 mmol) in 2-mL anhydrous DMF. The reaction was allowed to proceed at r.t. for two days under argon in the dark. Solvent was removed under reduced pressure to afford a yellowish solid. MS (ESI), calculated for C₈₆H₁₂₄N₁₀O₂₂ (M+Na)⁺: 1671.88. found 1671.90. Without further purification, the solid was dissolved in 4-mL DMF, followed by the addition of 1-mL piperidine at 0° C. The solution was stirred at 0° C. for 30 minutes and r.t. for 1 hour. After the solvent was removed under reduced pressure, the pale yellow solid was washed several times with DCM and subsequently with ddH₂O to afford a pale white solid. The reaction scheme is shown in FIG. 9. MS (ESI), calculated for C₇₁H₁₁₅N₁₀O₂₀ (M+Na)⁺: 1427.83. found 1427.84. ¹H-NMR (400 MHz, DMSO-d₆) δ (ppm): 8.59 (s, 1H), 8.26 (s, 1H), 8.05 (m, 2H), 7.92-7.84 (m, 2H), 7.08 (s, 1H), 6.44-6.30 (m, 2H), 6.20-6.14 (m, 1H), 5.99 (d, J=8 Hz, 1H), 5.59-5.53 (q, 1H), 5.44 (d, J=8 Hz, 1H), 5.35-5.08 (m, 2H), 4.64 (m, 1H), 4.36 (s, 1H), 4.28-4.07 (m, 3H), 3.99-3.89 (m, 2H), 3.82-3.79 (m, 4H), 3.72-3.66 (m, 2H), 3.63-3.50 (m, 1H), 3.43-3.29 (m, 5H), 3.29-3.22 (m, 3H), 3.2-3.16 (m, 3H), 3.10-2.92 (m, 9H), 2.82-2.57 (m, 2H), 2.37-2.35 (m, 2H), 2.27-2.14 (m, 3H), 2.04-1.95 (m, 3H), 1.8-0.8 (m, 55H).

### Synthesis of HA-Gly-Gly-Gly-Val-Cit-Glu-Asp-rapamycin

Twenty milligrams of sodium hyaluronate (Na-HA, 75 kDa, 0.05 mmol) and DMTMM (27.5 mg. 0.1 mmm01) were dissolved in 1-mL ddH₂O with 0.5 wt % sodium dodecyl sulfate (SDS). After the mixture was stirred at r.t. for 20 minutes, a solution of 42-O-(Gly-Gly-Gly-Val-Cit-Glu-Asp)-rapamycin (15 mg. 0.01 mmol) in 2-mL DMSO with 0.5 wt % SDS was added dropwise. The reaction was stirred at r.t. in the dark for two days. The organic solvent was removed by dialysis (10 kDa MWCO dialysis tubing) against ddH2O for 24 hours. The unreacted free drug produced a pale yellow precipitate, which was removed by filtration. The reaction scheme is shown in FIG. 9. The product, HA-Gly-Gly-Gly-Val-Cit-Glu-Asp-rapamycin conjugates, was further purified by washing several times with ddH₂O in a 20-mL centrifugal filter (PES, 10 kDa MWCO), and finally freeze-dried (Labconco 2.5 Plus FreeZone, Kansas City, Mo.).

### Characterization of HA-Gly-Gly-Gly-Val-Cit-Glu-Asp-rapamycin-UV/Vis Absorption Spectrum

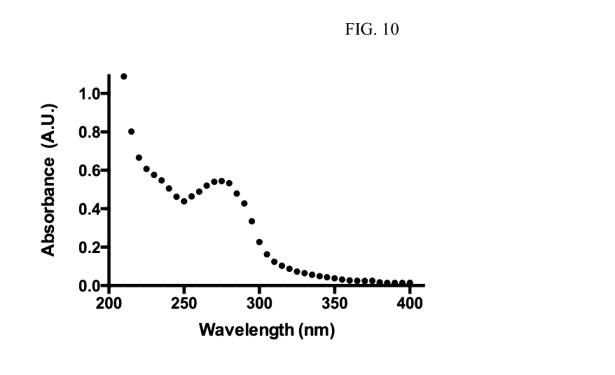

The UV/Vis absorption spectrum of a HA-Gly-Gly-Gly-Val-Cit-Glu-Asp-rapamycin solution in water was measured using a SpectraMax Plus spectrophotometer. A Na-HA solution at the same concentration was used as a reference. As shown in FIG. 10, the rapamycin content in the conjugates exhibited an absorbance band peak at 280 nm.

### Characterization of HA-Gly-Gly-Gly-Val-Cit-Glu-Asp-rapamycin-Gel Permeation Chromatography

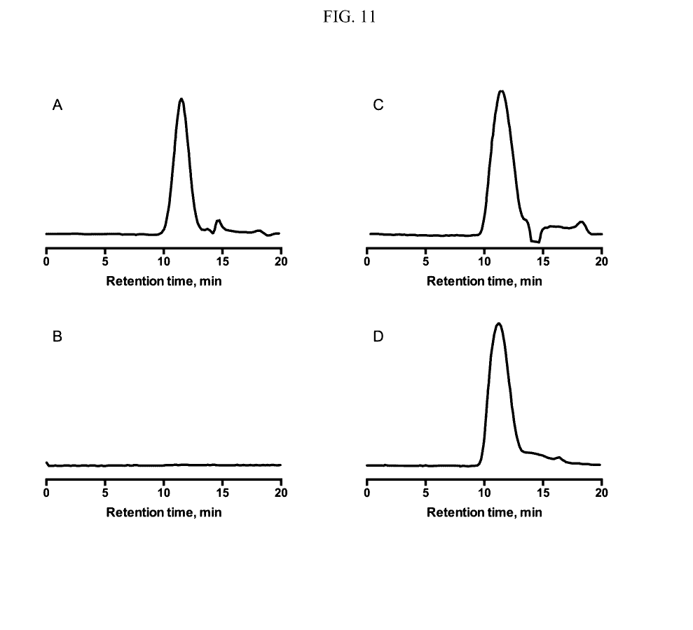

Gel permeation chromatography (GPC) was used to confirm the conjugation by comparing the elution time of Na-HA and HA-Gly-Gly-Gly-Val-Cit-Glu-Asp-rapamycin conjugates. GFC analysis was performed on a Shodex HQ-804 M column thermostated at 35° C. with 5-mM ammonium acetate (pH 5) as the mobile phase at a flow rate of 0.8 ml/min, and peaks were detected using a Shimadzu 2010CHT HPLC with a refractive index (RI) detector (Shimadzu RID-10A) and a UV/Vis detector at 210 and 280 nm. The conjugation was verified based on the equivalent retention times at approximately 10 min under both wavelengths of 210 and 280 nm, in contrast to no absorbance of Na-HA at 280 nm. FIG. 11 depicts chromatograms of Na-HA (A: 210 nm; B: 280 nm) and HA-Gly-Gly-Gly-Val-Cit-Glu-Asp-rapamycin conjugates (C: 210 nm; D: 280 nm).

In Vitro Drug Release

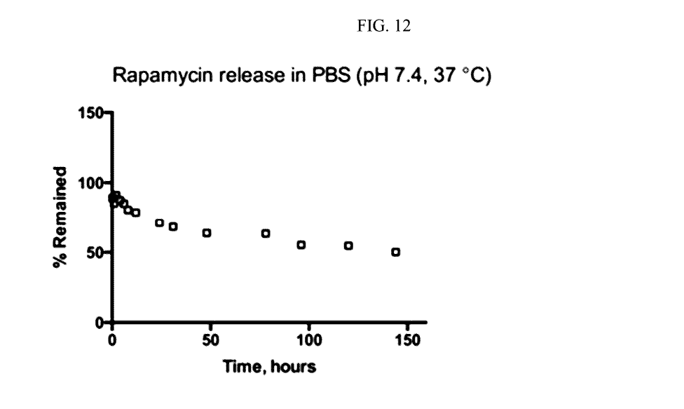

The in vitro release of rapamycin from HA-Gly-Gly-Gly-Val-Cit-Glu-Asp-rapamycin conjugates into PBS (pH 7.4) at 37° C. was monitored by a dialysis method using a SnakeSkin® pleated dialysis tubing (3500 MWCO). The PBS medium was changed once per day to maintain sink condition. Fifty-microliters of solution inside the dialysis tubing was withdrawn at predetermined time points, and analyzed using a SpectraMax Plus spectrophotometer with UV detection of rapamycin at 280 nm. The release profile was fitted using first-order kinetics, and the half-life and rate constant of release were 50 h and 0.014 h⁻¹, respectively. Results are shown in FIG. 12.

### Example 5

### Synthesis of HA-ethyldisulfide-Ala-rapamycin

### Synthesis of 2-hydroxyethyldisulfide-Fmoc-Ala ester

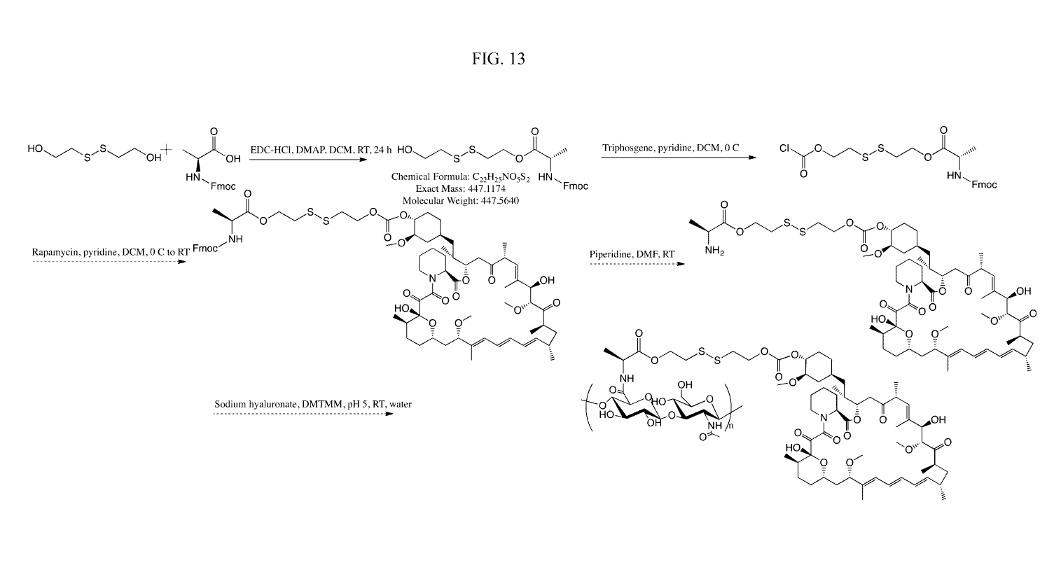

Fmoc-Ala-OH (500 mg, 1.6 mmol) was dissolved in 10 mL of dichloromethane (DCM). To this solution, 1-Ethyl-3-(3-dimethylaminopropyl)carbodiimide (EDC, 370 mg, 1.2 mmol) was added and stirred for 20 min. The solution becomes cloudy after 5 min. Then DMAP (12 mg, 5 mol %) and 2-hydroxyethyl disulfide (300 μL, 2.4 mmol) are added to the solution. The reaction is stirred at room temperature. Progress of the reaction was monitored by TLC. After completion of the reaction, water (50 mL) and EtOAc (40 mL) were added to the reaction mixture. EtOAc layer was separated and aqueous layer was re-extracted with EtOAc (20 mL×2). The EtOAc fractions were combined, dried over anhydrous MgSO₄, filtered, and the supernatant concentrated under reduced pressure to afford the crude title compound as a yellowish white solid. Crude product was purified over silica gel using EtOAc in hexane to afford pure product as a white solid. The synthesis scheme is shown in FIG. 13. MS (ESI), calculated for C₂₂H₂₅NO₅S₂ (M+Na)⁺: 470.1. found 470.2.

### Synthesis of 42-O-(ethyldisulfide-Fmoc-Ala)-rapamycin

Triphosgene (40 mg, 0.13 mmol) was completely dissolved in 3 mL of anhydrous DCM and cooled to −78° C. in an acetone-dry ice bath with stirring under Ar. 2-Hydroxyethyldisulfide-Fmoc-Ala ester (50 mg, 0.11 mmol) and anhydrous pyridine (9 μl. 0.11 mmol) in anhydrous DCM (5 mL) was slowly added to the reaction mixture over 1 hour under Ar. After the slow addition, the reaction mixture was allowed to stir for 30 min at −78° C. After 30 mins, the reaction mixture was allowed to warm up to room temperature and stirred for another 2 hours at room temperature under Ar. To this solution, added dropwise, a solution of rapamycin (100 mg, 0.11 mmol), and pyridine (9 μl, 0.11 mmol) in 5 mL of anhydrous DCM. The reaction was allowed to proceed at room temperature for 2 hours under argon in the dark. Saturated NH₄Cl (20 mL) was added to the reaction mixture. The organic layer was washed with saturated NH₄Cl (10 mL×2) and subsequently with ddH₂O (10 mL×3). The organic layer was dried over anhydrous MgSO4, filtered, and the supernatant concentrated under reduced pressure to afford the crude title compound as a white solid. Crude product was purified over silica gel using EtOAc in hexane to afford pure product as a white solid. The synthesis scheme is shown in FIG. 13. MS (ESI), calculated for C₇₄H₁₀₂N₂O₁₉S₂ (M+Na)⁺: 1409.6416. found 1409.6512.

### Synthesis of 42-O-(ethyldisulfide-Ala)-rapamycin

42-O-(ethyldisulfide-Ala)-rapamycin (100 mg, 0.08 mmol) was dissolved in 400 μL of DMF at 0° C. Piperidine (100 μL) was added to the reaction mixture and allowed to stir for 3 hours. After completion of the reaction, the solvent was removed under high vacuum with care. The crude product was purified over silica gel using EtOAc in hexane to afford pure product as a white solid. The synthesis scheme is shown in FIG. 13.

### Synthesis of HA-ethyldisulfide-Ala-rapamycin

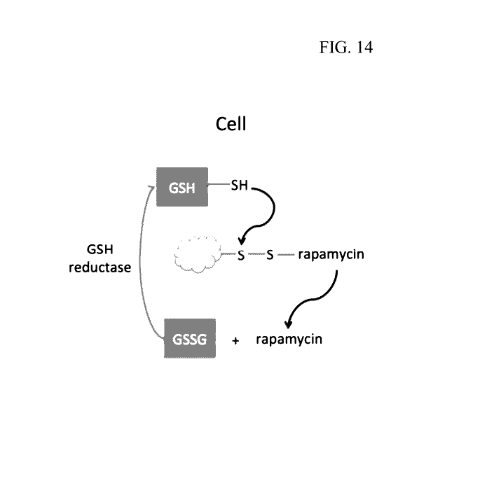

A scintillation vial was charged with hyaluronic acid (75 kDa) sodium salt (20 mg, 0.05 mmol based on COOH groups per disaccharide unit) in H₂O (1 mL) and 4-(4,6-Dimethoxy-1,3,5-triazin-2-yl)-4-methylmorpholin-4-ium chloride (DMTMM, 41 mg, 0.15 mmol) was added. The resulted solution was gently stirred for 15 minutes at room temperature. An aqueous solution (1 mL) of HA-ethyldisulfide-Ala-rapamycin (60 mg, 0.05 mmol) was then added to the reaction mixture and pH of the resulted solution was immediately adjusted to 5 using aqueous NaOH solution. The resulted reaction mixture was gently stirred for 48 hours at 37° C. The reaction mixture was dialyzed (10000 MWC) against NaCl (3×) and ultrapure water (3×) for 48 hours. The resulted solution was then filtered (0.2 μM filter) and lyophilized to afford the title conjugate as a white fluffy solid. Product was analyzed by ¹H NMR in deuterated water. Degree of substitution (DS) was calculated using the peaks at 1.96 ppm (3H, HA) and 1.38 ppm (3H, Ala CH₃). The synthesis scheme is shown in FIG. 13. The expected interaction between cellular thiols and HA-disulfide linker-rapamycin is shown in FIG. 14.

### Example 6

### HA-Carbamate-Rapamycin Conjugate Not Observed

### Example 6A

### Attempted synthesis of 42-O—(N-Fmoc 1,4-butanediamine carbamate)-rapamycin

42-O-(4-nitro-phenyloxycarbonyl)-rapamycin (10 mg, 9.3 μmol) was dissolved in 2 mL anhydrous DMF. To this solution N-Fmoc 1,4-butanediamine hydrobromide (6.28 mg, 18.6 μmol) was added and the mixture was stirred under argon protected from light for 24 hours, after which solvent was removed using reduced pressure. MS (ESI), calculated for C₇₁H₉₉N₃O₁₆ (M+H): 1250.70, (M+Na): 1272.70; No product mass observed.

### Example 6B

### Attempted synthesis of 42-O—(N-Fmoc 1,4-butanediamine carbamate)-rapamycin

42-O-(4-nitro-phenyloxycarbonyl)-rapamycin (10 mg, 9.3 μmol) was dissolved in 2 mL anhydrous DMF. To this solution N-Fmoc 1,4-butanediamine hydrobromide (3.14 mg, 9.3 μmol) was added and the mixture was stirred for 30 minutes. Pyridine (3.25 μL, 46.5 μmol) was added and the reaction mixture was stirred under argon protected from light for 24 hours, after which solvent was removed using reduced pressure. MS (ESI), calculated for C₇₁H₉₉N₃O₁₆ (M+H): 1250.70, (M+Na): 1272.70; No product mass observed.

### Example 6C

### Attempted synthesis of 42-O—(N-Fmoc 1,4-butanediamine carbamate)-rapamycin

42-O-(4-nitro-phenyloxycarbonyl)-rapamycin (10 mg, 9.3 μmol) was dissolved in 2 mL anhydrous DMF. To this solution N-Fmoc 1,4-butanediamine hydrobromide (6.28 mg, 18.6 μmol) was added and the mixture was stirred for 30 minutes. Pyridine (3.25 μL, 46.5 μmol) was added and the reaction mixture was stirred under argon protected from light for 36 hours, after which solvent was removed using reduced pressure. MS (ESI), calculated for C₇₁H₉₉N₃O₁₆ (M+H): 1250.70, (M+Na): 1272.70; No product mass observed.

### Example 6D

### Attempted synthesis of 42-O—(N-Fmoc 1,4-butanediamine carbamate)-rapamycin

42-O-(4-nitro-phenyloxycarbonyl)-rapamycin (10 mg, 9.3 μmol) was dissolved in 2 mL anhydrous DMF. To this solution N-Fmoc 1,4-butanediamine hydrobromide (6.28 mg, 18.6 μmol) was added and the mixture was stirred for 30 minutes. Pyridine (4.55 μL, 65.1 μmol) was added and the reaction mixture was stirred under argon protected from light for 24 hours, after which solvent was removed using reduced pressure. MS (ESI), calculated for C₇₁H₉₉N₃O₁₆ (M+H): 1250.70, (M+Na): 1272.70; No product mass found.

### Example 6E

### Attempted synthesis of 42-O—(N-Fmoc 1,4-butanediamine carbamate)-rapamycin

42-O-(4-nitro-phenyloxycarbonyl)-rapamycin (10 mg, 9.3 μmol) was dissolved in 2 mL anhydrous DMF. To this solution N-Fmoc 1,4-butanediamine hydrobromide (9.42 mg, 27.9 μmol) was added and the mixture was stirred for 30 minutes. Pyridine (9.75 μL, 0.14 mmol) was added and the reaction mixture was stirred under argon protected from light for 24 hours, after which solvent was removed using reduced pressure. MS (ESI), calculated for C₇₁H₉₉N₃O₁₆ (M+H): 1250.70, (M+Na): 1272.70; No product mass found.

### Example 7

### HA-Ester-Rapamycin Conjugates Not Observed

### Example 7A

### Attempted synthesis of 42-O-(3-amino-4-methoxy-benzoate)-rapamycin

Rapamycin (30 mg, 0.033 mmol) was dissolved in 1 mL anhydrous DCM. To this solution, N-Fmoc 3-amino-4-methoxy-benzoyl chloride (47 mg, 0.116 mmol) dissolved into 1 mL anhydrous DCM was added. Hünig's base (45.7 μL, 0.264 mmol) dissolved in 1 mL anhydrous DCM was added dropwise, and the reaction mixture was stirred for 2 hours under argon protected from light. MS (ESI) calculated for C₅₉H₈₆N₂O₁₅ (M+H)⁺: 1063.60; No product mass observed.

### Example 7B

### Attempted synthesis of 42-O-(3-amino-4-methoxy-benzoate)-rapamycin

N-Fmoc 3-amino-4-methoxy-benzoyl chloride (52.4 mg, 0.128 mmol) was dissolved in 2.5 mL of anhydrous DCM immediately after oxalyl chloride/DCM removal under reduced pressure. Rapamycin (33.3 mg, 0.037 mmol) dissolved in 1 mL anhydrous DCM and added dropwise over 1 minute. Hünig's base (38.1 μL, 0.220 mmol) was added, and the reaction mixture was stirred under argon protected from light. Monitoring by TLC (35/65 Hexane/Acetone, Silica gel 60 F₂₅₄) showed total Rapamycin degradation within 10 minutes.

### Example 7C

### Attempted synthesis of 42-O-(3-amino-4-methoxy-benzoate)-rapamycin

N-Fmoc 3-amino-4-methoxy-benzoyl chloride (52.4 mg, 0.128 mmol) was dissolved in 2.5 mL of anhydrous DCM immediately after oxalyl chloride/DCM removal under reduced pressure. Rapamycin (33.3 mg, 0.037 mmol) dissolved in 1 mL anhydrous DCM and added dropwise over 1 minute, and the reaction mixture was stirred under argon protected from light. Monitoring by TLC (35/65 Hexane/Acetone, Silica gel 60 F₂₅₄) showed total Rapamycin degradation within 10 minutes.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

### REFERENCES

Chan J S, Robertson H A, Friesen H G. The purification and characterization of ovine placental lactogen. Endocrinology. 1976; 98(1):65-76.
Tzircotis G, Thorne R F, Isacke C M. Chemotaxis towards hyaluronan is dependent on CD44 expression and modulated by cell type variation in CD44-hyaluronan binding. J Cell Sci. 2005; 118(Pt 21):5119-28.
Olsson E, Honeth G, Bendahl P O, Saal L H, Gruvberger-Saal S, et al. CD44 isoforms are heterogeneously expressed in breast cancer and correlate with tumor subtypes and cancer stem cell markers. BMC Cancer. 2011; 11:418.
Misra S, Heldin P, Hascall V C, Karamanos N K, Skandalis S S, et al. Hyaluronan-CD44 interactions as potential targets for cancer therapy. FEBS J. 2011; 278(9):1429-43.
Ghosh S C, Neslihan Alpay S, Klostergaard J. CD44: a validated target for improved delivery of cancer therapeutics. Expert Opin Ther Targets. 2012; 16(7):635-50.
Peer D, Margalit R. Loading mitomycin C inside long circulating hyaluronan targeted nano-liposomes increases its antitumor activity in three mice tumor models. Int J Cancer. 2004; 108(5):780-9.
Platt V M, Szoka F C, Jr. Anticancer therapeutics: targeting macromolecules and nanocarriers to hyaluronan or CD44, a hyaluronan receptor. Mol Pharm. 2008; 5(4):474-86.
Ferrari M. Cancer nanotechnology: opportunities and challenges. Nat Rev Cancer. 2005; 5(3): 161-71.
Li C, Wallace S. Polymer-drug conjugates: recent development in clinical oncology. Adv Drug Deliv Rev. 2008; 60(8):886-98.
Shakya A K, Nandakumar K S. Applications of polymeric adjuvants in studying autoimmune responses and vaccination against infectious diseases. J R Soc Interface. 2013; 10(79):20120536.
Liechty W B, Kryscio D R, Slaughter B V, Peppas N A. Polymers for drug delivery systems. Annu Rev Chem Biomol Eng. 2010; 1:149-73.
Pasut G, Veronese F M. PEG conjugates in clinical development or use as anticancer agents: an overview. Adv Drug Deliv Rev. 2009; 61(13):1177-88.
Jain A, Jain S K. PEGylation: an approach for drug delivery. A review. Crit Rev Ther Drug Carrier Syst. 2008; 25(5):403-47.
Vicent M J, Dieudonne L, Carbajo R J, Pineda-Lucena A. Polymer conjugates as therapeutics: future trends, challenges and opportunities. Expert Opin Drug Deliv. 2008; 5(5):593-614.
Vaidya A, Agarwal A, Jain A, Agrawal R K, Jain S K. Bioconjugation of polymers: a novel platform for targeted drug delivery. Curr Pharm Des. 2011; 17(11):1108-25.
Du W, Gerald D, Perruzzi C A, Rodriguez-Waitkus P, Enayati L, et al. Vascular tumors have increased p70 S6-kinase activation and are inhibited by topical rapamycin. Lab Invest. 2013.
Ge Y, Chen J. Mammalian target of rapamycin (mTOR) signaling network in skeletal myogenesis. J Biol Chem. 2012; 287(52):43928-35.
LoRusso P M. Mammalian target of rapamycin as a rational therapeutic target for breast cancer treatment. Oncology. 2013; 84(1):43-56.
Chong Z Z, Shang Y C, Maiese K. Cardiovascular disease and mTOR signaling. Trends Cardiovasc Med. 2011; 21(5):151-5.
Kamada Y, Funakoshi T, Shintani T, Nagano K, Ohsumi M, et al. Tor-mediated induction of autophagy via an Apg1 protein kinase complex. J Cell Biol. 2000; 150(6):1507-13.
Zhao Y, Sun Y. Targeting the mTOR-DEPTOR pathway by CRL E3 ubiquitin ligases: therapeutic application. Neoplasia. 2012; 14(5):360-7.
Aoki M, Blazek E, Vogt P K. A role of the kinase mTOR in cellular transformation induced by the oncoproteins P3k and Akt. Proc Natl Acad Sci USA. 2001; 98(1):136-41.
Gu J, Ruppen M E, Cai P. lipase-catalyzed regioselective esterification of rapamycin: synthesis of temsirolimus (CCI-779). Org Lett. 2005; 7(18):3945-8.
Hioki A, Wakasugi A, Kawano K, Hattori Y, Maitani Y. Development of an in vitro drug release assay of PEGylated liposome using bovine serum albumin and high temperature. Biol Pharm Bull. 2010; 33(9):1466-70.
Doane T L, Chuang C H, Hill R J, Burda C. Nanoparticle zeta-potentials. Acc Chem Res. 2012; 45(3):317-26.
Yanez J A, Forrest M L, Ohgami Y, Kwon G S, Davies N M. Pharmacometrics and delivery of novel nanoformulated PEG-b-poly(epsilon-caprolactone) micelles of rapamycin. Cancer Chemother Pharmacol. 2008; 61(1): 133-44.
Taryn R. Bagby S C, Shaofeng Duan, Sharadvi Thati, Daniel J. Aires, M. Larid Forrest. Impact of Molecular Weight on Lymphatic Drainage of a Biopolymer-Based Imaging Agent. Pharmaceutics. 2012; 4:276-96.
Wolny P M, Banerji S, Gounou C, Brisson A R, Day A J, et al. Analysis of CD44-hyaluronan interactions in an artificial membrane system: insights into the distinct binding properties of high and low molecular weight hyaluronan. J Biol Chem. 2010; 285(39):30170-80.
Myckatyn T M, Ellis R A, Grand A G, Sen S K, Lowe J B, 3rd, et al. The effects of rapamycin in murine peripheral nerve isografts and allografts. Plast Reconstr Surg. 2002; 109(7):2405-17.
Hussein O, Tiedemann K, Murshed M, Komarova S V. Rapamycin inhibits osteolysis and improves survival in a model of experimental bone metastases. Cancer Lett. 2012; 314(2):176-84.
Lennon F E, Singleton P A. Role of hyaluronan and hyaluronan-binding proteins in lung pathobiology. Am J Physiol Lung Cell Mol Physiol. 2011; 301(2):L137-47.
Jackson D G. Biology of the lymphatic marker LYVE-1 and applications in research into lymphatic trafficking and lymphangiogenesis. APMIS. 2004; 112(7-8):526-38.
Leung K. Hyaluronan conjugated with IR-783. Molecular Imaging and Contrast Agent Database (MICAD). Bethesda (Md.) 2004.
Wendt M K, Molter J, Flask C A, Schiemann W P. In vivo dual substrate bioluminescent imaging. J Vis Exp. 2011(56).
Dumont F J, Su Q. Mechanism of action of the immunosuppressant rapamycin.
Life Sci. 1996; 58(5):373-95.
Kay J E, Kromwel L, Doe S E, Denyer M. Inhibition of T and B lymphocyte proliferation by rapamycin. Immunology. 1991; 72(4):544-9.
Law B K. Rapamycin: an anti-cancer immunosuppressant? Crit Rev Oncol Hematol. 2005; 56(1):47-60.
Gupta, P. B., C. L. Chaffer, and R. A. Weinberg, Cancer stem cells: mirage or reality? Nat Med, 2009. 15(9): p. 1010-2.
Medema, J. P., Cancer stem cells: the challenges ahead. Nat Cell Biol, 2013. 15(4): p. 338-44.
Al-Hajj, M., et al., Prospective identification of tumorigenic breast cancer cells. Proc Natl Acad Sci USA, 2003. 100(7): p. 3983-8.
Bonnet, D. and J. E. Dick, Human acute myeloid leukemia is organized as a hierarchy that originates from a primitive hematopoietic cell. Nat Med, 1997. 3(7): p. 730-7.
Kim, C. F., et al., Identification of bronchioalveolar stem cells in normal lung and lung cancer. Cell, 2005. 121(6): p. 823-35.
O'Brien, C. A., et al., A human colon cancer cell capable of initiating tumour growth in immunodeficient mice. Nature, 2007. 445(7123): p. 106-10.
Singh, S. K., et al., Identification of human brain tumour initiating cells. Nature, 2004. 432(7015): p. 396-401.
Hanahan, D. and R. A. Weinberg, Hallmarks of cancer: the next generation. Cell, 2011. 144(5): p. 646-74.
Leth-Larsen, R., et al., Functional heterogeneity within the CD44 high human breast cancer stem cell-like compartment reveals a gene signature predictive of distant metastasis. Mol Med, 2012. 18: p. 1109-21.
Li, X., et al., Intrinsic resistance of tumorigenic breast cancer cells to chemotherapy. J Natl Cancer Inst, 2008. 100(9): p. 672-9.
Moriwaki, K., et al., Combination use of anti-CD133 antibody and SSA lectin can effectively enrich cells with high tumorigenicity. Cancer Sci, 2011. 102(6): p. 1164-70.
Courtney, K. D., R. B. Corcoran, and J. A. Engelman, The PI3K pathway as drug target in human cancer. J Clin Oncol, 2010. 28(6): p. 1075-83.
Lim, H. J., P. Crowe, and J. L. Yang, Current clinical regulation of PI3K/PTEN/Akt/mTOR signalling in treatment of human cancer. J Cancer Res Clin Oncol, 2014.
Zhou, J., et al., Activation of the PTEN/mTOR/STAT3 pathway in breast cancer stem-like cells is required for viability and maintenance. Proc Natl Acad Sci USA, 2007. 104(41): p. 16158-63.
Yilmaz, O. H., et al., Pten dependence distinguishes haematopoietic stem cells from leukaemia-initiating cells. Nature, 2006. 441(7092): p. 475-82.
Miller, T. W., et al., Mutations in the phosphatidylinositol 3-kinase pathway: role in tumor progression and therapeutic implications in breast cancer. Breast Cancer Res, 2011. 13(6): p. 224.
Davis, N. M., et al., Deregulation of the EGFR/PI3K/PTEN/Akt/mTORC1 pathway in breast cancer: possibilities for therapeutic intervention. Oncotarget, 2014. 5(13): p. 4603-50.
Yocum, D. E., Cyclosporine, FK-506, rapamycin, and other immunomodulators. Rheum Dis Clin North Am, 1996. 22(1): p. 133-54.
Sehgal, S. N., Rapamune (Sirolimus, rapamycin): an overview and mechanism of action. Ther Drug Monit, 1995. 17(6): p. 660-5.
Saunders, R. N., M. S. Metcalfe, and M. L. Nicholson, Rapamycin in transplantation: a review of the evidence. Kidney Int, 2001. 59(1): p. 3-16.
Cejka, D., et al., Mammalian target of rapamycin signaling is crucial for joint destruction in experimental arthritis and is activated in osteoclasts from patients with rheumatoid arthritis. Arthritis Rheum, 2010. 62(8): p. 2294-302.
Keever-Taylor, C. A., et al., Rapamycin enriches for CD4(+) CD25(+) CD27(+) Foxp3(+) regulatory T cells in ex vivo-expanded CD25-enriched products from healthy donors and patients with multiple sclerosis. Cytotherapy, 2007. 9(2): p. 144-57.
Prevel, N., et al., Beneficial role of rapamycin in experimental autoimmune myositis. PLoS One, 2013. 8(11): p. e74450.
Esposito, M., et al., Rapamycin inhibits relapsing experimental autoimmune encephalomyelitis by both effector and regulatory T cells modulation. J Neuroimmunol, 2010. 220(1-2): p. 52-63.
Zhang, Z., et al., Low dose rapamycin exacerbates autoimmune experimental uveitis. PLoS One, 2012. 7(5): p. e36589.
Morath, C., et al., Sirolimus in renal transplantation. Nephrol Dial Transplant, 2007. 22 Suppl 8: p. viii61-viii65.

What is claimed and desired to be secured by Letters Patent is as follows:

1. A pharmaceutical composition comprising:
a drug conjugate comprising
an mTOR inhibitor selected from the group consisting of rapamycin, everolimus, temsirolimus, and deforolimus;
hyaluronic acid, and
a linker coupling the hyaluronic acid to the mTOR inhibitor, wherein the linker comprises an ester, a carbonate, or a carbamate coupled to the mTOR inhibitor.

2. The composition of claim 1, wherein the linker further comprises a hydrazide, ester, carbonate, ether, carbamate, carbonyl, urea, alkyl or amine coupled to the hyaluronic acid.

3. The composition of claim 1, wherein the linker comprises the ester, the carbonate or the carbamate comprising a hindered or electron rich labile bond.

4. The composition of claim 3, wherein the linker comprises an aromatic group.

5. The composition of claim 4, wherein the aromatic group is benzene.

6. The composition of claim 5, wherein the linker comprises 3-amino-4-methoxy-benzoate, and wherein a 3-amino group forms an amide with the hyaluronic acid.

7. The composition of claim 1, wherein the linker comprises the carbamate.

8. The composition of claim 7, wherein the linker comprises a diamine.

9. The composition of claim 8, wherein the linker comprises 1,4-butanediamine.

10. The composition of claim 1, wherein the linker comprises a biologically labile linkage that is preferentially cleaved inside cells, wherein said cleavage results in spontaneous labiality of the ester, carbamate, carbonate or amide.

11. The composition of claim 10, wherein the biologically labile linkage is a biologically labile peptide sequence.

12. The composition of claim 11, wherein the biologically labile peptide sequence includes at least one sequence selected from the group consisting of Phe-Lys, Val-Lys, Ala-Lys, Phe-Phe-Lys, Ala-Phe-Lys, Gly-Phe-Lys, Ac-Phe-Lys, HCO-Phe-Lys, Val-Cit, Phe-Cit, Leu-Cit, Ile-Cit, Trp-Cit, Phe-Arg(NO₂)₂, and Phe-Arg(Ts).

13. The composition of claim 12, wherein the peptide sequence comprises Val-Cit.

14. The composition of claim 13, wherein the peptide sequence comprises Gly-Gly-Gly-Val-Cit-Glu-Asp [SEQ. ID. NO: 1].

15. The composition of claim 10, wherein the biologically labile sequence is a biologically labile disulfide sequence.

16. The composition of claim 15, wherein the linker comprises the ester, the carbonate or the carbamate and the cleavage inside the cell results in formation of a 5 or 6 member ring able to induce labiality in the ester, carbonate or carbamate.

17. The composition of claim 15, wherein the biologically labile sequence comprises an ethyl or propyl thiol group.

18. The composition of claim 17, wherein the biologically labile sequence comprises ethyldisulfide.

19. The composition of claim 15, wherein the linker comprises an amino acid having an amino group, and the amino group provides an amide linkage with hyaluronic acid.

20. The composition of claim 10, wherein the linker comprises the carbonate or the carbamate.

21. The composition of claim 1, wherein an amino acid provides an amino group for an amide linkage with hyaluronic acid.

22. The composition of claim 1, wherein the conjugate is a nanoparticle configured for preferential uptake into a tumor or lymph node.

23. The composition of claim 22, where the nanoparticle has a size between 9 and 100 nm.

24. A therapeutic method comprising:
administering a therapeutically effective amount of the composition of claim 1 to a subject in need thereof.

25. A pharmaceutical composition comprising:
a drug conjugate comprising
an mTOR inhibitor selected from the group consisting of BGT226, SF1126, BEZ235, Gedatolisib and SF1101;
hyaluronic acid, and
a linker coupling the hyaluronic acid to the mTOR inhibitor, wherein the linker comprises an ester, a carbonate, or a carbamate coupled to the mTOR inhibitor.
